(19) United States
(12) Patent Application Publication
(10) Pub. No.: US 2025/0009879 A1
(43) Pub. Date: Jan. 9, 2025

(54) NATURAL KILLER CELL-SPECIFIC CHIMERIC ANTIGEN RECEPTOR AND USE THEREOF

(71) Applicants: THE ASAN FOUNDATION, Seoul (KR); UNIVERSITY OF ULSAN FOUNDATION FOR INDUSTRY COOPERATION, Ulsan (KR)

(72) Inventors: Hun Sik KIM, Seoul (KR); Hyo Jin PARK, Namyangju-si (KR); Eunbi YI, Seoul (KR)

(73) Assignees: THE ASAN FOUNDATION, Seoul (KR); UNIVERSITY OF ULSAN FOUNDATION FOR INDUSTRY COOPERATION, Ulsan (KR)

(21) Appl. No.: 18/712,346

(22) PCT Filed: Nov. 14, 2022
(86) PCT No.: PCT/KR2022/017855

§ 371 Date: May 22, 2024

(30) Foreign Application Priority Data
Nov. 22, 2021 (KR) 10-2021-0161425
Nov. 9, 2022 (KR) 10-2022-0149000

(51) Int. Cl. A61K 39/00; C07K 14/705; C07K 14/725; C07K 16/28; C07K 16/30; C07K 16/32; C12N 5/0783

(52) U.S. Cl. CPC A61K 39/4631; A61K 39/4613; A61K 39/464406; A61K 39/464411; A61K 39/464412; A61K 39/464413; A61K 39/464419; A61K 39/464428; A61K 39/464468; A61K 39/464482; A61K 39/464493; A61K 39/464495; C07K 14/705; C07K 14/70503; C07K 14/7051; C07K 14/70517; C07K 14/70521; C07K 16/2803; C07K 16/2827; C07K 16/2866; C07K 16/2878; C07K 16/2884; C07K 16/2893; C07K 16/3007; C07K 16/3069; C07K 16/3084; C07K 16/32; C12N 5/0646; C07K 2317/622; C07K 2319/02; C07K 2319/03; C12N 2510/00

(57) ABSTRACT

A chimeric antigen receptor (CAR), including an antigen-binding domain, a CD8 or CD28 hinge domain, a DAP10 cytoplasmic domain, a 2B4 cytoplasmic domain, and a CD3z cytoplasmic domain, where the CAR is expressed in natural killer (NK) cells. A method for treating cancer, including administering a therapeutically effective amount of a composition containing a NK cell that expresses the CAR, or a therapeutically effective amount of a cellular therapeutic agent containing a NK cell that expresses the CAR, to a subject in need thereof.

### TECHNICAL FIELD

The present invention relates to a natural killer cell-specific chimeric antigen receptor, natural killer cells that express the same, and a use thereof.

The present invention claims priority to and the benefit of Korean Patent Application No. 10-2021-0161425, filed on Nov. 22, 2021, and Korean Patent Application No. 10-2022-0149000, filed on Nov. 9, 2022, the disclosures of which are incorporated herein by reference in their entirety.

### BACKGROUND ART

Cancers are among the diseases that cause some of the largest numbers of deaths in modem people. They are diseases caused by changes in normal cells due to genetic mutations or the like, and are defined as malignant tumors that do not follow the differentiation, proliferation, and growth patterns of normal cells. Particularly, cancer is characterized by “uncontrolled cell growth,” and in this case, due to abnormal cell growth, a cell mass called a tumor is formed and infiltrates surrounding tissues, and in severe cases, also metastasizes to other organs of the body. Cancer is an intractable chronic disease that is fundamentally cured even with surgery, radiation, and drug therapy in many cases, causes pain to patients, and ultimately leads to death. Particularly, recently, due to the increase in elderly population and environmental deterioration, cancer cases have increased globally by 5% or more every year, and according to the WHO report, it is estimated that, within the next 25 years, the number of people with cancer will increase to 30 million, 20 million of whom will die from cancer.

Drug therapy for cancer, that is, with anticancer agents, generally involves cytotoxic compounds that treat cancer by attacking and killing cancer cells, and damages not only cancer cells but also normal cells, resulting in severe side effects. Therefore, to reduce side effects, targeted anticancer agents have been developed. However, although these targeted anticancer agents can reduce side effects, they have shown limitations in that they have a high probability of developing resistance. Accordingly, recently, there has been a rapidly growing interest in immune-anticancer agents that use the immune system in the body to reduce problems caused by toxicity and resistance. As an example of such an immune-anticancer agent, an immune checkpoint inhibitor that specifically binds to PD-L1 on the surface of cancer cells to inhibit binding of T cells with PD-1 and activate the T cells, resulting in attacking cancer cells, has been developed. However, since the type of cancer on which such an immune checkpoint inhibitor is effective is limited, there is an urgent need to develop new immune checkpoint inhibitors that are equally effective in treating various types of cancer.

Recently, research has been actively conducted on anticancer immune cell therapy, which induces cancer cell death by directly injecting immune cells that attack cancer into the body.

Natural killer cells (NK cells), one of the representative immune cells used in anticancer immune cell therapy, are a type of cytotoxic lymphocytes responsible for innate immune responses, have large granules in the cytoplasm morphologically, and account for 5 to 20% of lymphocytes in the blood. NK cells have various immune receptors on their surface and thus are able to distinguish between cancer cells and normal cells, so they have an advantage of being able to immediately detect and remove cancer cells. Particularly, since NK cells can not only inhibit the development, proliferation, and metastasis of cancer cells and virus-infected cells, but also effectively remove cancer stem cells, they can prevent the recurrence of cancer caused by cancer stem cells, and therefore are considered effective anticancer immune cells. In addition, in various clinical studies, when NK cells isolated from relatives or normal people are introduced into patients, the immune rejection is extremely small compared to other immune cells, and therefore their potential use as a cellular therapeutic agent is receiving attention.

Meanwhile, a chimeric antigen receptor (CAR) is an artificial receptor designed to deliver antigen specificity to immune cells, and consists of antigen-specific components, transmembrane components and intracellular components selected to activate immune cells and provide specific immunity. Recently, cancer immunotherapy using cells to which a CAR-encoding gene is introduced has been attempted. The most representative immunotherapy is a method of treating cancer by collecting T cells from a patient, introducing CAR-encoding gene, amplifying T cells expressing CAR and injecting the CAR-T cells into a patient, that is, a method using CAR-T cells. However, although CAR-T cells have excellent anticancer effects, they have side effects such as a cytokine release syndrome and neurotoxicity, and there is a limitation in that CAR-T cells have to be derived from autologous T cells to prevent immune rejection. Furthermore, there are problems in that the process of producing autologous CAR-T is very complicated, the production cost is high, and the process still has immune side effects. To overcome these problems, research on allogeneic off-the-shelf CAR-T is progressing, but there is no CAR-T developed to solve these problems yet.

Therefore, recently, an NK cell-based treatment “CAR-NK,” which has a lower risk of immune side effects compared to CAR-T and allows an allogeneic cellular therapeutic agent to be easily produced, has been attracting attention. NK cells have a wider range of cancer cell recognition compared to other immune cells, and can be produced in allogeneic cells without separate genetic manipulation. Particularly, since there is almost no risk of graft-versus-host diseases caused by immune rejection, there is an advantage in that not only autologous cells but also allogeneic cells can be used. In addition, NK cells are more efficient than T cells in that they can be provided as off the shelf and do not need to be customized to individual patients, and are also expected to be applicable to CAR-T-resistant carcinomas that occur when the expression of antigens (tumor-associated antigens, AACs) is reduced by cancer cells.

That is, a CAR-NK treatment using NK cells can be mass-produced, resulting in reduced treatment costs, and has low immunogenicity, and can therefore be considered a solid cancer treatment and an alternative treatment for reducing cytokine side effects. However, most research on chimeric antigen receptors so far has been based on T cell components, and it is difficult to apply it to NK cells. Therefore, there is a need to develop new chimeric antigen receptors tailored to the characteristics of NK cells.

### DISCLOSURE

### Technical Problem

The present invention was invented to address the above problems, and was completed by confirming that a chimeric antigen receptor (CAR) including a specific combination of natural killer (NK) cell-specific activation receptor domains can effectively activate NK cells, enhancing the activity of killing target cells (e.g., cancer cells).

Therefore, the present invention is directed to providing an NK cell-specific CAR.

The present invention is also directed to providing NK cells that express the CAR.

The present invention is also directed to providing a nucleic acid molecule that encodes the CAR, and/or an expression vector including the same.

The present invention is also directed to providing cells transformed by the expression vector.

The present invention is also directed to providing a pharmaceutical composition for preventing or treating cancer, which includes NK cells expressing the CAR as an active ingredient.

The present invention is also directed to providing a cellular therapeutic agent for preventing or treating cancer, which includes NK cells expressing the CAR as an active ingredient.

The present invention is also directed to providing a method of preparing a NK cell-specific CAR, which includes:
(S1) transforming the expression vector to an isolated cell; and
(S2) culturing the transformed cell obtained in (S1).

However, technical problems to be solved in the present invention are not limited to the above-described problems, and other problems which are not described herein will be fully understood by those of ordinary skill in the art from the following descriptions.

### Technical Solution

The present invention provides an NK cell-specific CAR, which includes (i) an antigen-binding domain; (ii) a CD8 or CD28 hinge domain; (iii) a DAP10 cytoplasmic domain; (iv) a 2B4 cytoplasmic domain; and (v) a CD3z cytoplasmic domain, wherein the CAR is expressed in NK cells.

In one embodiment of the present invention, the antigen-binding domain may be a tumor antigen-specific antibody or an antigen-binding fragment thereof, but the present invention is not limited thereto.

In another embodiment of the present invention, the CAR may satisfy one or more of the following characteristics, but the present invention is not limited thereto:
(a) the CD8 hinge domain includes the amino acid sequence of SEQ ID NO: 2;
(b) the CD28 hinge domain includes the amino acid sequence of SEQ ID NO: 3;
(c) the DAP10 cytoplasmic domain includes the amino acid sequence of SEQ ID NO: 7;
(d) the 2B4 cytoplasmic domain includes the amino acid sequence of SEQ ID NO: 8; and
(e) the CD3z cytoplasmic domain includes the amino acid sequence of SEQ ID NO: 9.

In still another embodiment of the present invention, the CAR may further include a CD28 transmembrane domain, but the present invention is not limited thereto.

In yet another embodiment of the present invention, the CD28 transmembrane domain may include the amino acid sequence of SEQ ID NO: 4, but the present invention is not limited thereto.

In yet another embodiment of the present invention, the CAR may further include one or more selected from the group consisting of a DAP10 extracellular domain and a DAP10 transmembrane domain, but the present invention is not limited thereto.

In yet another embodiment of the present invention, the CAR may satisfy one or more of the following characteristics, but the present invention is not limited thereto:
(f) the DAP10 extracellular domain includes the amino acid sequence of SEQ ID NO: 5; and
(g) the DAP10 transmembrane domain includes the amino acid sequence of SEQ ID NO: 6.

In yet another embodiment of the present invention, the CAR may further include a signal peptide, but the present invention is not limited thereto.

In yet another embodiment of the present invention, the signal peptide may be a CD8 signal peptide, but the present invention is not limited thereto.

In yet another embodiment of the present invention, the tumor antigen may be one or more selected from the group consisting of CD19, TAG72, an interleukin 13 receptor alpha-2 subunit (IL13Rα2), CD52, CD33, CD20, TSLPR, CD22, CD30, GD3, CD171, a neural cell adhesion molecule (NCAM), a folate binding protein (FBP), a Lewis-Y antigen (Le(Y)), a prostate stem cell antigen (PSCA), a prostate-specific membrane antigen (PSMA), a carcinoembryonic antigen (CEA), human epidermal growth factor receptor 2 (HER2), mesothelin, hyaluronate receptor variant 6 (CD44v6), B7-H3, Glypican-3, receptor tyrosine kinase-like orphan receptor 1 (ROR1), survivin, folate receptor 1 (FOLR1), Wilm's tumor 1 (WT1), vascular endothelial growth factor 2 (VEGFR2), EGFR, and KRAS, but the present invention is not limited thereto.

In yet another embodiment of the present invention, the antigen-binding fragment may be selected from the group consisting of scFv, (scFv)₂, Fab, Fab′, and F(ab′)₂, but the present invention is not limited thereto.

In yet another embodiment of the present invention, the tumor antigen-specific antibody or an antigen-binding fragment thereof may include a heavy chain variable region and a light chain variable region as below, but the present invention is not limited thereto:
(i) a heavy chain variable region that includes heavy chain CDR1 including the amino acid sequence of SEQ ID NO: 26, heavy chain CDR2 including the amino acid sequence of SEQ ID NO: 27, and heavy chain CDR3 including the amino acid sequence of SEQ ID NO: 28; and
(ii) a light chain variable region that includes light chain CDR1 including the amino acid sequence of SEQ ID NO: 29, light chain CDR2 including the amino acid sequence of SEQ ID NO: 30, and light chain CDR3 including the amino acid sequence of SEQ ID NO: 31.

In yet another embodiment of the present invention, the tumor antigen-specific antibody or an antigen-binding fragment thereof may include the amino acid sequence of SEQ ID NO: 1, but the present invention is not limited thereto.

In addition, the present invention provides NK cells expressing the NK cell-specific CAR according to the present invention. That is, the present invention provides CAR-NK cells including (expressing) the CAR.

In one embodiment of the present invention, the NK cells may satisfy one or more characteristics selected from the group consisting of as below, but the present invention is not limited thereto:
(a) an increase in secretion amount of one or more selected from the group consisting of MIP-1α, granzyme B, and INF-γ upon antigen recognition;
(b) an increase in activity of one or more selected from the group consisting of an AKT signal and an ERK signal upon antigen recognition; and
(c) an increase in degranulation level upon antigen recognition.

In addition, the present invention provides a nucleic acid molecule which encodes the NK cell-specific CAR according to the present invention.

In addition, the present invention provides an expression vector which includes the nucleic acid molecule.

In addition, the present invention provides isolated cells into which the expression vector is introduced.

In addition, the present invention provides a pharmaceutical composition for preventing or treating cancer, which includes NK cells expressing the NK cell-specific CAR according to the present invention as an active ingredient. Preferably, an antigen-binding domain of the CAR is a tumor antigen-specific antibody or an antigen-binding fragment thereof.

The present invention also provides a cellular therapeutic agent for preventing or treating cancer, which includes NK cells expressing the NK cell-specific CAR according to the present invention as an active ingredient.

The present invention also provides a method of preventing or treating cancer, which includes administering NK cells expressing the NK cell-specific CAR to a subject in need thereof.

The present invention also provides a use of the NK cell-specific CAR, a nucleic acid molecule encoding the CAR, an expression vector including the nucleic acid molecule, cells into which the expression vector is introduced, and/or NK cells expressing the CAR for preventing or treating cancer.

The present invention also provides a use of the NK cell-specific CAR, a nucleic acid molecule encoding the CAR, an expression vector including the nucleic acid molecule, cells into which the expression vector is introduced, and/or NK cells expressing the CAR for preparing a drug for treating cancer. The drug may be a cellular therapeutic agent for treating cancer.

In one embodiment of the present invention, the cancer may be one or more selected from the group consisting of colorectal cancer, rectal cancer, colon cancer, thyroid cancer, oral cancer, pharynx cancer, larynx cancer, cervical cancer, brain cancer, lung cancer, ovarian cancer, bladder cancer, kidney cancer, liver cancer, pancreatic cancer, prostate cancer, skin cancer, tongue cancer, breast cancer, uterine cancer, stomach cancer, bone cancer, lymphoma, blood cancer, squamous cell carcinoma, adenocarcinoma of the lung, peritoneal cancer, skin melanoma, ocular melanoma, perianal cancer, esophageal cancer, small intestine cancer, endocrine cancer, parathyroid cancer, adrenal cancer, soft tissue sarcoma, urethral cancer, gastrointestinal cancer, glioblastoma, endometrial cancer, salivary gland cancer, vulvar cancer, and head and neck cancer, but the present invention is not limited thereto.

The present invention also provides a method of preparing an NK cell-specific CAR, which includes (S1) introducing the expression vector into isolated cells; and (S2) culturing the expression vector-introduced cells.

The present invention also provides a method of preparing NK cells expressing an NK cell-specific CAR, which includes introducing the expression vector into isolated NK cells.

### Advantageous Effects

The present invention relates to an NK cell-specific CAR (NK-CAR), which is completed by confirming that the NK-CAR including a specific combination of NK cell-specific activation receptor-derived domains can effectively activate NK cells and enhance the activity of killing target cells. Specifically, the present inventors prepared three types of NK cell-specific CARs consisting of the combination of specific domains, and confirmed that, in the NK cells expressing the NK-CAR, the production of cytokine and granzyme B responding to target cells or AKT and ERK activation are further enhanced, and degranulation is promoted, resulting in exhibiting better cytolytic activity. That is, since the NK-CAR according to the present invention can enhance cytolytic activity by strongly inducing the activation of NK cells, it can be used in immunotherapy for treating various diseases, including cancer. Particularly, CAR-based anticancer treatment using NK cells cannot cause cytokine release syndrome or neurotoxicity, but can use autologous and allogeneic NK cells, so it has an advantage of fewer side effects compared to CAR-T. Therefore, the NK cells that express the NK-CAR according to the present invention are expected to be used as a cellular therapeutic agent with low side effects and a maximized drug treatment effect.

### DESCRIPTION OF DRAWINGS

FIG. 1 shows the structures of chimeric antigen receptors NK-CAR1, NK-CAR2, NK-CAR3, and control 2^{nd} generation CAR according to the present invention.

FIG. 2A shows the results of confirming the proportions of GFP- and NK-CAR-expressing cells through flow cytometry three days after infecting NKL cells with an NK-CAR retrovirus according to the present invention.

FIG. 2B shows the results of confirming the proportions of GFP- and NK-CAR-expressing cells through flow cytometry, after isolating GFP-expressing cells from NKL cells and culturing them three days after retroviral infection.

FIG. 2C shows the results of confirming the proportions of GFP- and NK-CAR-expressing cells through flow cytometry three days after infecting NK-92 cells with an NK-CAR retrovirus according to the present invention.

FIG. 2D shows the results of confirming the proportions of GFP- and NK-CAR-expressing cells through flow cytometry, after isolating GFP-expressing cells from NK-92 cells and culturing them three days after retroviral infection.

FIG. 3A shows the results of measuring cytolytic activity against a CD19-positive REH cell line of an empty vector, control 2^{nd} CAR, or NK-CAR-expressing NKL cells according to the present invention through the Europium assay (hereinafter, EV: empty vector).

FIGS. 3B and 3C show the results of measuring cytolytic activity against a CD19-positive REH cell line (FIG. 3B) and a RAMOS cell line (FIG. 3C) of an empty vector, control 2^{nd} CAR, or NK-CAR-expressing NK92 cells according to the present invention through the Europium assay.

FIGS. 4A and 4B show the results of measuring the levels of MPI-lu (FIG. 4A) and granzyme B (FIG. 4B) secreted from NKL cells through ELISA, after co-culturing each of an empty vector, control 2^{nd} CAR, and CAR-NK-expressing NKL cells according to the present invention with a CD19-positive REH cell line.

FIG. 5A shows the results of confirming the degree of the activation of AKT and ERK signals through Western blotting after stimulating NKL cells or NK-CAR-expressing NKL cells with CD19-positive REH cells for 2 or 5 minutes.

FIG. 5B shows the results of confirming the degree of the activation of AKT and ERK signals through Western blotting after stimulating NKL cells or NK-CAR-expressing NKL cells with a recombinant human CD19 Fc chimera for 2 or 5 minutes.

FIG. 6 shows the results of measuring the degree of degranulation of NK92 cells after co-culturing an empty vector, control 2^{nd} CAR, or NK-CAR-expressing NK92 cells according to the present invention with CD19-positive cells (REH or Ramos).

FIG. 7 shows the results of measuring the proportions of Interferon-γ positive NK92 cells after co-culturing an empty vector, control 2^{nd} CAR, or NK-CAR-expressing NK92 cells according to the present invention with CD19-positive cells (REH or Ramos).

FIG. 8 shows one example of a preferable amino acid sequence of NK-CAR1.

FIG. 9 shows one example of a preferable amino acid sequence of NK-CAR2.

FIG. 10 shows one example of a preferable amino acid sequence of NK-CAR3.

### MODES OF THE INVENTION

The present invention relates to a natural killer cell (NK cell)-specific chimeric antigen receptor (CAR), completed by confirming that CAR including a specific combination of NK cell-specific activation receptor domains is effective in activation of NK cells and is able to induce cancer cell-specific death.

Specifically, in one embodiment of the present invention, retroviral vectors of three types of NK-CAR genes according to the present invention and a T cell-based CAR (control) gene were manufactured (Example 1), and CARs were expressed in NK cells (NKL cells and NK92 cells) using retroviruses into which the vectors were introduced (Example 2).

In another embodiment of the present invention, as a result of evaluating the cancer cytolytic activity of NK-CAR-expressing NKL cells and NK92 cells, it was confirmed that both of the NK-CAR-expressing NKL cells and the NK92 cells more effectively caused the death of target cells (cancer cells) compared to the control (Example 3).

In still another embodiment of the present invention, as a result of evaluating the cytokine secretion ability of NK-CAR-expressing NKL cells, when stimulating the NK-CAR-expressing NKL cells with target cells (cancer cells), it was confirmed that the levels of MIP-la and granzyme B secretion were further increased compared to a control (Example 4).

In yet another embodiment of the present invention, as a result of evaluating the degree of the activation of AKT and ERK signals according to the recognition of a target antigen of NK-CAR-expressing NKL cells, when stimulating the NK-CAR-expressing NKL cells with cancer cells that expressed a target antigen protein or target antigen, it was confirmed that levels of AKT and ERK phosphorylation were further increased compared to a control (Example 5).

In yet another embodiment of the present invention, it was confirmed that NK-CAR-expressing NK92 cells had a higher degree of degranulation according to target antigen recognition compared to a control (Example 6).

In yet another embodiment of the present invention, it was confirmed that NK-CAR-expressing NK92 cells had a higher degree of cytokine production according to target antigen recognition compared to a control (Example 7).

That is, the NK-CAR according to the present invention can more effectively activate NK cells in response to a target and enhance cytolytic activity thereof, and therefore is expected to be used as a novel immunotherapy means for preventing and treating various diseases, including cancer.

Hereinafter, the present invention will be described in detail.

The present invention provides an NK-CAR, which is expressed in NK cells.

In the present invention, “chimeric antigen receptor (CAR)” refers to a synthetic receptor that can target a specific antigen. The CAR according to the present invention includes an antigen-binding domain, a transmembrane domain, and a cytoplasmic domain to form the structure of the receptor, and preferably, further includes a hinge region.

In the present invention, “antigen-binding domain” refers to a protein or polypeptide domain that can specifically recognize and bind a target antigen. In the present invention “antigen” refers to a polypeptide, compound, or other material that can specifically bind to a humoral immune mediator such as an antibody, or a cellular immune mediator such as a T cell receptor.

The antigen according to the present invention is not limited to a specific type, and one of ordinary skill in the art may suitably select an appropriate antigen and an antigen-binding domain that can specifically bind to the antigen according to a purpose. For example, when an infectious disease caused by bacteria or viruses is to be prevented or treated, an antigen-binding domain specific to the bacterium- or virus-specific protein may be selected.

Preferably, the antigen is a tumor antigen. Tumor antigens includes a tumor-specific antigen expressed only in cancer cells, and a tumor-associated antigen which is expressed in normal cells and also expressed particularly in cancer cells with high frequency, or has higher activity. For example, the tumor antigen may be a surface protein that is expressed only in cancer cells, or expressed with a higher frequency in cancer cells. The tumor antigen is not limited to a specific type, but is preferably selected from the group consisting of CD19, TAG72, an interleukin 13 receptor alpha-2 subunit (IL13Rα2), CD52, CD33, CD20, TSLPR, CD22, CD30, GD3, CD171, a neural cell adhesion molecule (NCAM), a folate-binding protein (FBP), a Lewis-Y antigen (Le(Y)), a prostate stem cell antigen (PSCA), a prostate-specific membrane antigen (PSMA), a carcinoembryonic antigen (CEA), human epidermal growth factor receptor 2 (HER2), mesothelin, hyaluronate receptor variant 6 (CD44v6), B7-H3, glypican-3, receptor tyrosine kinase like orphan receptor 1 (RORI), survivin, folate receptor 1 (FOLR1), Wilm's tumor 1 (WT1), vascular endothelial growth factor 2 (VEGFR2), an epidermal growth factor receptor (EGFR), and KRAS.

In the present invention, “hinge region” refers to a part that is located between an antigen-binding domain and a transmembrane domain to serve as a flexible linker, and is also called a “spacer.” When the antigen-binding domain is bound with an antigen, the hinge region allows the antigen-binding domain to be properly located to form a stable bond with an antigen. For example, the hinge region has the purpose of extending the antigen-binding domain from the cell membrane of a cell expressing a CAR.

In the present invention, “transmembrane domain (TM)” refers to any polypeptide or oligopeptide that serves to link extracellular and cytoplasmic domains through the cell membrane.

The transmembrane domain may pass through the cell membrane such that the antigen-binding domain of the CAR may be located on a cell surface (outside the cell), and the cytoplasmic domain may be located in cells. That is, the transmembrane domain serves as a support for the CAR and at the same time connects an antigen-binding domain (or a hinge domain) and a cytoplasmic domain.

In the present invention, “cytoplasmic domain or cytoplasmic signaling domain (CYP)” refers to a polypeptide or oligopeptide inside the cell membrane. The cytoplasmic domain receives a signal delivered by an antigen-binding domain to serve to deliver the signal into cells expressing the CAR. The signal is delivered into cells to trigger activation or inhibition in a biological process. The type of cytoplasmic domain is not particularly limited as long as it can deliver a signal that can induce the activation of NK cells when an antigen-binding domain is bonded with an antigen. In the present invention, the cytoplasmic domain may include one or more intracellular co-stimulatory domains. The co-stimulatory domains are located in an extracellular or intracellular part of the CAR, and serve to deliver signals to CAR-expressing cells. That is, the co-stimulatory domains contribute to induction of a sufficient reaction (activation) of NK cells according to the binding of a target antigen. The co-stimulatory domains may be selected from, for example, CD27, CD28, 4-1BB, OX40, CD30, CD40, PD-1, ICOS, lymphocyte function-associated antigen-1 (LFA-1), CD2, CD7, LIGHT, NKG2C, and/or B7-H3-derived domains, but the present invention is not limited thereto.

Each domain of the CARs according to the present invention may selectively be connected by a short oligopeptide or polypeptide linker. When a target antigen is bound to an extracellular antigen-binding domain, the length or type of the linker is not particularly limited as long as the linker can induce NK cell activation by an intracellular domain, and a linker known in the art may be applied without limitation.

Each domain of the CAR according to the present invention may include the above-described domains as well as modified forms of the domains. Here, the modification may be performed by substituting, deleting or adding one or more amino acids in the amino acid sequences of wild-type antibodies and domains without modifying the functions of the antibodies and domains. Typically, the substitution may be performed with an alanine, or by conservative amino acid substitution without affecting charges, polarity, or hydrophobicity of the entire proteins.

There is no limit to the type of immune cells (T cells, NK cells, NKT cells, macrophages, etc.) in which the CAR according to the present invention can be expressed. Preferably, the CAR of the present invention, as an NK cell-specific CAR, is expressed in NK cells. Preferably, the CAR according to the present invention is located on the cell membrane of NK cells. That is, the CAR according to the present invention is specialized for the functional activation of NK cells, and when bonding with a target antigen, it activates a signaling pathway that triggers the target cell death function of NK cells, ultimately inducing NK cells to specifically kill target cells. The CAR according to the present invention can be expressed not only in autologous cells but also in allogeneic cells of an administration subject.

The hinge domain of the CAR according to the present invention may be CD8, CD28, IgG1, IgG4, and/or killer immunoglobulin-like receptor (KIR)-derived domains, but the present invention is not limited thereto, and a hinge domain conventionally used in the art may be applied without limitation.

Preferably, in the present invention, the hinge domain may be a CD8 (preferably, CD8α) hinge domain. The CD8 hinge domain preferably includes the amino acid sequence of SEQ ID NO: 2, and more preferably the amino acid sequence of SEQ ID NO: 2, but the present invention is not limited thereto. Variants of the amino acid sequence are included in the scope of the present invention. That is, the CD8 hinge domain may include an amino acid sequence having 70% or more, more preferably 80% or more, even more preferably 90% or more, and most preferably 95% or more sequence homology with the amino acid sequence represented by SEQ ID NO: 2.

In addition, in the present invention, the hinge domain of CAR may be a CD28 hinge domain. Preferably, the CD28 hinge domain may include the amino acid sequence of SEQ ID NO: 3, or may be encoded by a polypeptide consisting of the amino acid sequence of SEQ ID NO: 3, but the present invention is not limited thereto. That is, the CD28 hinge domain may include an amino acid sequence having 70% or more, more preferably 80% or more, even more preferably 90% or more, and most preferably 95% or more sequence homology with the amino acid sequence represented by SEQ ID NO: 3.

The cytoplasmic domain of CAR according to the present invention may be DAP10, 2B4, and/or CD3z-derived domains, but the present invention is not limited thereto, and any that can deliver signals that activate NK cells by the stimulation of a target antigen may be applied without limitation.

In the present invention, the DAP10 cytoplasmic domain may include the amino acid sequence of SEQ ID NO: 7, or may be encoded by a polypeptide consisting of the amino acid sequence of SEQ ID NO: 7, but the present invention is not limited thereto. That is, the DAP10 cytoplasmic domain may include an amino acid sequence having 70% or more, more preferably 80% or more, even more preferably 90% or more, and most preferably 95% or more sequence homology with the amino acid sequence represented by SEQ ID NO: 7.

In addition, according to the present invention, the 2B4 cytoplasmic domain may include the amino acid sequence of SEQ ID NO: 8, or may be encoded by a polypeptide consisting of the amino acid sequence of SEQ ID NO: 8, but the present invention is not limited thereto. That is, the 2B4 cytoplasmic domain may include an amino acid sequence having 70% or more, more preferably 80% or more, even more preferably 90% or more, and most preferably 95% or more sequence homology with the amino acid sequence represented by SEQ ID NO: 8.

In addition, in the present invention, the CD3z (CD3ζ) cytoplasmic domain may include the amino acid sequence of SEQ ID NO: 9, or may be encoded by a polypeptide consisting of the amino acid sequence of SEQ ID NO: 9, but the present invention is not limited thereto. That is, the CD3z cytoplasmic domain may include an amino acid sequence having 70% or more, more preferably 80% or more, even more preferably 90% or more, and most preferably 95% or more sequence homology with the amino acid sequence represented by SEQ ID NO: 9.

Preferably, the CAR according to the present invention may further include a CD28 transmembrane domain. In addition, the CD28 transmembrane domain may include the amino acid sequence of SEQ ID NO: 4, or may be encoded by a polypeptide consisting of the amino acid sequence of SEQ ID NO: 4, but the present invention is not limited thereto. That is, the CD28 transmembrane domain may include an amino acid sequence having 70% or more, more preferably 80% or more, even more preferably 90% or more, and most preferably 95% or more sequence homology with the amino acid sequence represented by SEQ ID NO: 4.

Preferably, the CAR according to the present invention may further include a DAP10 extracellular domain. In addition, the DAP10 extracellular domain may include the amino acid sequence of SEQ ID NO: 5, or may be encoded by a polypeptide consisting of the amino acid sequence of SEQ ID NO: 5, but the present invention is not limited thereto. That is, the DAP10 extracellular domain may include an amino acid sequence having 70% or more, more preferably 80% or more, even more preferably 90% or more, and most preferably 95% or more sequence homology with the amino acid sequence represented by SEQ ID NO: 5.

Preferably, the CAR according to the present invention may further include a DAP10 transmembrane domain. In addition, the DAP10 transmembrane domain may include the amino acid sequence of SEQ ID NO: 6, or may be encoded by a polypeptide consisting of the amino acid sequence of SEQ ID NO: 6, but the present invention is not limited thereto. That is, the DAP10 transmembrane domain may include an amino acid sequence having 70% or more, more preferably 80% or more, even more preferably 90% or more, and most preferably 95% or more sequence homology with the amino acid sequence represented by SEQ ID NO: 6.

Preferably, the CAR according to the present invention may further include a signal peptide. In the present invention, a “signal peptide” is an amino acid sequence located at the N-terminus of a protein, and serves to induce a newly synthesized protein to move to a specific location, such as the endoplasmic reticulum (ER). In the present invention, the signal peptide may be derived from a molecule selected from, for example, CD8 (CD8α), GM-CSF receptor α, Ig-κ, or IgG1 heavy chain, but the present invention is not limited thereto. Preferably, in the present invention, the signal peptide may be a CD8 signal peptide. In addition, the CD8 signal peptide may include the amino acid sequence of SEQ ID NO: 10, or may be encoded by a polypeptide consisting of the amino acid sequence of SEQ ID NO: 10, but the present invention is not limited thereto. That is, the DAP10 transmembrane domain may include an amino acid sequence having 70% or more, more preferably 80% or more, even more preferably 90% or more, and most preferably 95% or more sequence homology with the amino acid sequence represented by SEQ ID NO: 10. The signal peptide according to the present invention is preferably located at the N-terminus of a recombinant protein (CAR), and is used to move the protein on a cell surface. When the recombinant protein is expressed, a part of the sequence may be cut out, leaving only some amino acids, or the entire sequence may be cut out and thus the signal peptide sequence part may not exist.

Preferably, the antigen-binding domain is the antigen-specific antibody or a fragment thereof (preferably, an antigen-binding fragment of the antibody). In one embodiment of the present invention, the antigen-binding domain is an antigen-binding domain specifically binding to a tumor antigen, and a tumor antigen-specific antibody or an antigen-binding fragment thereof.

In the present invention, “antibody” refers to an immunoglobulin molecule that immunologically has reactivity with a specific antigen (epitope), and includes all of a polyclonal antibody, a monoclonal antibody, and a functional fragment thereof. In addition, the term may include forms that are produced by genetic engineering, such as a chimeric antibody (e.g., a humanized murine antibody) and a heterogeneous antibody (e.g., a bispecific antibody). The antibody includes heavy chain and/or light chain variable region(s) (VH, heavy chain variable region; VL, light chain variable region). The variable region is a primary structure, and includes a part that forms an antigen binding site of the antibody molecule, and the antibody of the present invention may be composed of a partial fragment including the variable region.

The term “epitope” refers to a specific three-dimensional molecular structure within an antigen molecule to which an antibody specifically binds.

In the present invention, “fragment” of an antibody refers to a (functional) fragment that retains the antigen-binding function of an antibody, and preferably, an antigen-binding fragment of the antibody. The term “fragment” is used to include scFv, (scFv)₂, Fab, Fab′, and F(ab′)₂ as well as a nanobody fragment. This definition of “fragment” is well known in the art. Preferably, the antigen-binding fragment is scFv.

Among the antibody fragments, Fab is a structure that has light chain and heavy chain variable regions and a light chain constant region and a first constant region (CH1) of a heavy chain, and has one antigen binding site. Fab′ differs from Fab in that it has a hinge region containing one or more cysteine residues at the C-terminus of the heavy chain CH1 domain. F(ab′)2 antibody is produced when cysteine residues in the hinge region of Fab′ form a disulfide bond. Fv is the smallest antibody fragment having only a heavy chain variable region and a light chain variable region. Two-chain Fv is formed by connecting a heavy chain variable region and a light chain variable region using a non-covalent bond. In single-chain Fv (scFv), generally, a heavy chain variable region and a light chain variable region are covalently connected using a peptide linker, or directly connected to the C-terminus, thereby forming a dimer-like structure such as two-chain Fv.

More specifically, a “single-chain Fv” or “scFv” antibody fragment refers to a protein in which the variable regions of the light and heavy chains of an antibody are connected by a linker consisting of a peptide sequence in which about 15 amino acids are linked. These domains exist in a single polypeptide chain. An Fv polypeptide may further include a polypeptide linker between a VH domain and a VL domain to allow the scFv to form the desired structure for antigen binding. The term “Fv” fragment used herein refers to an antibody fragment containing complete antibody recognition and binding sites. This region consists of a dimer in which one heavy chain variable domain and one light chain variable domain are tightly and substantially covalently associated, for example, with scFv.

Antibodies exhibit antigen specificity depending on changes in sequence of a variable region. The variable region of an antigen binding site is divided into a framework region (FR) with low variability and a complementarity determining region (CDR) with high variability, and both the heavy chain and the light chain have three CDR sites divided into CDR1, 2, and 3, and four FR sites. The CDR is a site in the variable region of an antibody that provides binding specificity to an antigen. The CDRs of each chain are typically named CDR1, CDR2, and DR3 sequentially from the N-terminus, and identified by chains on which specific CDRs are located.

In one embodiment of the present invention, the tumor antigen-specific antibody or antigen-binding fragment thereof according to the present invention may include the following heavy chain variable region and/or light chain variable region:
(i) a heavy chain variable region that includes heavy chain CDR1 including the amino acid sequence of SEQ ID NO: 26, heavy chain CDR2 including the amino acid sequence of SEQ ID NO: 27, and/or heavy chain CDR3 including the amino acid sequence of SEQ ID NO: 28; and
(ii) a light chain variable region that includes light chain CDR1 including the amino acid sequence of SEQ ID NO: 29, light chain CDR2 including the amino acid sequence of SEQ ID NO: 30, and/or light chain CDR3 including the amino acid sequence of SEQ ID NO: 31.

The CDRs of the present invention may include biological equivalents thereof. That is, each CDR may include an amino acid sequence having 70% or more, more preferably 80% or more, even more preferably 90% or more, and most preferably 95% or more sequence homology with the indicated amino acid sequence.

In another embodiment of the present invention, the tumor antigen-specific antibody or antigen-binding fragment thereof according to the present invention may include the amino acid sequence of SEQ ID NO: 1, or may be encoded by a polypeptide consisting of the amino acid sequence of SEQ ID NO: 1, but the present invention is not limited thereto. That is, the antibody or fragment thereof may include an amino acid sequence having 70% or more, more preferably 80% or more, even more preferably 90% or more, and most preferably 95% or more sequence homology with the amino acid sequence represented by SEQ ID NO: 1. Most preferably, the tumor antigen-specific antibody or antigen-binding fragment thereof may be anti-CD19 antibody or an scFv fragment thereof.

The NK-CAR according to the present invention may include the combination of an antigen-binding domain, a CD8 domain, a CD28 domain, a DAP10 domain, a 2B4 domain, and/or a CD3z domain. More specifically, the NK cell-specific CAR according to the present invention may include an antigen-binding domain, a CD8 or CD28 hinge domain, a DAP10 cytoplasmic domain, a 2B4 cytoplasmic domain, and a CD3z cytoplasmic domain. Preferably, the CAR may further include a CD28 transmembrane domain. The CAR may further include one or more selected from the group consisting of a DAP10 extracellular domain and a DAP10 transmembrane domain.

In an exemplary embodiment of the present invention, the NK-CAR may include the combination of an antigen-binding domain, a CD8 domain, a DAP10 domain, a 2B4 domain, and a CD3z domain; the combination of an antigen-binding domain, a CD28 domain, a DAP10 domain, a 2B4 domain, and CD3z; or the combination of an antigen-binding domain, a CD8 domain, a CD28 domain, a DAP10 domain, a 2B4 domain, and a CD3z domain.

In another exemplary embodiment of the present invention, the NK-CAR may sequentially include an antigen-binding domain, a CD8 hinge domain, a DAP10 extracellular domain, a DAP10 transmembrane domain, a DAP10 cytoplasmic domain, a 2B4 cytoplasmic domain, and a CD3z cytoplasmic domain (NK-CAR1). In another embodiment of the present invention, the NK cell-specific CAR may sequentially include an antigen-binding domain, a CD28 hinge domain, a DAP10 extracellular domain, a DAP10 transmembrane domain, a DAP10 cytoplasmic domain, a 2B4 cytoplasmic domain, and a CD3z cytoplasmic domain (NK-CAR2). In another embodiment of the present invention, the NK cell-specific CAR may sequentially include an antigen-binding domain, a CD8 hinge domain, a CD28 transmembrane domain, a DAP10 cytoplasmic domain, a 2B4 cytoplasmic domain, and a CD3z cytoplasmic domain (NK-CAR3).

The CAR according to the present invention may also include the above-described polypeptide domain as well as a biological equivalent thereof. For example, to further improve the antigen recognition ability and/or intracellular signaling ability of the CAR, an additional change may be made to the amino acid sequence of each domain.

In addition, preferably, the NK-CAR according to the present invention may include an amino acid sequence having 70% or more, more preferably 80% or more, even more preferably 90% or more, and most preferably 95% or more sequence homology with any one indicated amino acid sequence of SEQ ID NOs: 23 to 25.

A polypeptide (or a nucleic acid molecule) represented by a specific sequence herein may include the corresponding sequence as well as a biological equivalent thereof. That is, when considering variations with the biologically equivalent activity of the polypeptide (nucleic acid molecule), the polypeptide (or nucleic acid molecule) of one aspect is interpreted as also including a sequence having substantial identity with a sequence allocated to a sequence number. Specifically, a polypeptide (nucleic acid molecule) including an amino acid sequence (nucleotide sequence) represented by a specific sequence number is not limited only to a corresponding amino acid sequence (nucleotide sequence), and a variant of the amino acid sequence (nucleotide sequence) is included in the scope of the present invention. The polypeptide molecule (nucleic acid molecule) consisting of the amino acid sequence (nucleotide sequence) represented by a specific sequence number of the present invention is the concept includes functional equivalents of the polypeptide molecule (nucleic acid molecule) constituting the same, for example, variants in which some amino acid sequence (nucleotide sequence) of a polypeptide molecule (nucleic acid molecule) is modified by deletion, substitution, or insertion, but can have the same functional effect as a corresponding polypeptide (nucleic acid molecule). Specifically, the polypeptide (nucleic acid molecule) described in the present invention may include an amino acid sequence (nucleotide sequence) having 70% or more, more preferably 80% or more, even more preferably 90% or more, and most preferably 95% or more sequence homology with the amino acid sequence represented by a specific sequence number. For example, the polypeptide (nucleic acid molecule) of the present invention includes polypeptides (nucleic acid molecules) having 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100% sequence homology. The “% sequence homology” for the polypeptide (nucleic acid molecule) is determined by comparing two optimally aligned sequences and a comparative region, and a part of the polypeptide sequence (nucleotide sequence) in the comparative region may include an addition or deletion (i.e., a gap) compared to the reference sequence (not including an addition or deletion) for the optical alignment of two sequences.

In addition, the present invention provides cells that express NK-CAR according to the present invention. The cells may be in vitro or in vivo cells, and may be prepared as (or derived from) autologous cells and/or homologous cells (i.e., allogeneic cells) of an individual requiring the cells, or xenogenic cells thereof. Preferably, the cells may be prepared as autologous cells and/or synchronous cells of an individual requiring the same. In an exemplary embodiment of the present invention, the cells express the CAR in the cell membrane thereof. The cells may be immune cells such as T cells, NK cells, NKT cells, or macrophages. Preferably, the cells are NK cells. That is, the present invention provides CAR-NK cells that express the CAR according to the present invention. Since, among the immune cells, the NK cells have most instant and powerful killing ability, the CAR-NK cells of the present invention may be used in potent immunotherapy in the treatment of a disease such as cancer.

In the present invention, “natural killer cells (NK cells)” are a type of cytotoxic lymphocytes derived from bone marrow. NK cells account for 5 to 20% of all lymphocytes, and are responsible for innate immunity, and although there is no major histocompatibility complex or antibody on their surfaces, they provide immediate immune responses against virus-infected cells, cancer cells, or other modified cells. The NK cells include NK cells isolated from an individual as well as NK cells cultured or modified therefrom, or commercially available NK cells can also be used. That is, the NK cells of the present invention are not limited to specific types, and any having molecular characteristics and biological activity the same as or similar to those of NK cells are sufficient. In one embodiment, the NK cells may be selected from, for example, HANK1, NKL, NK92, NK-YS, YT, NOI-90, and NK101. These cells are exemplary, and the present invention is not limited thereto. The CAR-expressing NK cells according to the present invention may be chimeric antigen receptor natural killer cells (CAR-NK cells).

The CAR-expressing NK cells according to the present invention may be activated more rapidly or to a higher level than NK cells that do not express the corresponding CAR when a target is recognized by the receptor, and may more rapidly or effectively kill target cells.

For example, the CAR-expressing NK cells according to the present invention may increase in production and/or secretion of cytokines when recognizing a target antigen. That is, the NK-CAR-expressing NK cells of the present invention may exhibit a higher production and/or secretion level of cytokines compared to NK cells that do not express NK-CAR when recognizing a target. Preferably, the cytokines are secreted from activated NK cells to induce the death of target cells. Preferably, the cytokines are cytokines that can induce an anticancer effect by inhibiting the growth, proliferation, migration, and/or metastasis of cancer cells. The cytokines are not limited to specific types, and are preferably macrophage inflammatory protein-1α (MIP-1α) and/or interferon-γ (IFN-γ). However, those of ordinary skill in the art may recognize that the cytokines of the present invention may include cytokines related to the cell death mechanism of NK cells as well as the above-described cytokines.

The CAR-expressing NK cells according to the present invention may increase production and/or secretion of granzyme when recognizing a target antigen. That is, the level of the production and/or secretion of granzyme in the NK-CAR-expressing NK cells of the present invention may be higher than those of the NK cells that do not express NK-CAR when recognizing a target. The granzyme includes both of granzyme A and B.

In addition, the NK cells according to the present invention may increase in the activity of a signaling pathway associated with the activation of NK cells and/or target cell killing ability when recognizing a target antigen. Preferably, the signaling pathway is a signaling pathway that is associated with anticancer activity of NK cells (i.e., a signaling pathway for inhibiting cancer cell death or growth). That is, the NK-CAR-expressing NK cells according to the present invention may have higher activity of the signaling pathway than the NK cells that do not express NK-CAR when recognizing a target. Representative examples of the signaling pathway include an AKT signaling pathway and an ERK signaling pathway. That is, the intracellular AKT and/or ERK phosphorylation may increase when the CAR-expressing NK cells recognize a target antigen, resulting in activating AKT and/or ERK signaling pathway(s).

In addition, the degree (level) of degranulation of the CAR-expressing NK cells according to the present invention may increase when a target antigen is recognized. That is, degranulation of the NK-CAR-expressing NK cells of the present invention may more actively occur compared to the NK cells that do not express NK-CAR when recognizing a target. As evidence of this, the CAR-expressing NK cells according to the present invention may be further increased in CD107a level compared to the NK cells that do not express CAR when recognizing a target.

In addition, the present invention provides a nucleic acid molecule (i.e., polynucleotide) that encodes the NK-CAR according to the present invention.

The term “polynucleotide” used herein refers to an oligomer or polymer that includes deoxyribonucleic acid (DNA) and ribonucleic acid (RNA), and generally includes two or more combined nucleotides or nucleotide derivatives, which are bound with each other by a phosphodiester bond. Polynucleotides also include a nucleotide analogue, or DNA and RNA derivatives that include a “backbone” bond other than a phosphodiester bond, for example, a phosphotriester bond, a phosphoamidate bond, a phosphorothioate bond, a thioester bond, or a peptide bond (peptide nucleic acid). Polynucleotides include single-stranded and/or double-stranded polynucleotides, for example, DNA and RNA, as well as analogues of either RNA or DNA.

That is, the nucleic acid molecule according to the present invention includes genes that encode domains constituting the CAR according to the present invention. Each gene may be directly connected to the N- or C-terminus of another gene, and connected by a linker sequence.

Preferably, the nucleic acid molecule according to the present invention may include one or more base sequences selected from SEQ ID NOs: 11 to 22. Like the above-described polypeptide, the polynucleotide according to the present invention includes a functional equivalent thereof.

Accordingly, the nucleic acid molecule according to the present invention may include a base sequence having 70% or more, more preferably 80% or more, even more preferably 90% or more, and most preferably 95% or more sequence homology with one or more base sequences selected from the group consisting of SEQ ID NOs: 11 to 22.

Specifically, in the nucleic acid molecule according to the present invention, the site encoding a CD8 hinge domain may include a base sequence represented by SEQ ID NO: 12 or 13. In addition, the site encoding a CD28 hinge domain may include a base sequence represented by SEQ ID NO: 14. In addition, the site encoding a CD28 transmembrane domain may include the base sequence represented by SEQ ID NO: 15. In addition, the sites encoding a DAP10 extracellular domain, a transmembrane domain, and a cytoplasmic domain may sequentially include the base sequence represented by SEQ ID NOs: 16, 17, and 18, respectively.

In addition, the 2B4 cytoplasmic domain may include the base sequence represented by SEQ ID NO: 19. In addition, the CD3z cytoplasmic domain may include the base sequence represented by SEQ ID NO: 20.

An appropriate antigen-binding domain-coding gene may be selected depending on a purpose by one of ordinary skill in the art. For example, when the NK-CAR is for anticancer treatment, one of ordinary skill in the art may use a tumor antigen-specific antibody known in the art or a gene encoding a fragment thereof, or a newly manufactured gene sequence. In one embodiment of the present invention, the nucleic acid molecule of the NK cell-specific CAR may include a polynucleotide encoding a CD-19-specific antibody or a fragment thereof. Preferably, the polynucleotide or a fragment thereof may include the base sequence of SEQ ID NO: 11.

In addition, the nucleic acid molecule according to the present invention may include a site encoding a CD8 signal peptide (preferably, including the base sequence of SEQ ID NO: 21).

In addition, the present invention provides an expression vector including the nucleic acid molecule according to the present invention. That is, the present invention provides a recombinant vector that includes the nucleic acid molecule according to the present invention.

In the present invention, “recombinant vector” refers to a vector that can express a peptide or protein encoded by a heterologous nucleic acid inserted into the vector, and preferably, a vector manufactured to express a target protein (in the present invention, NK-specific CAR). “Vector” refers to any vehicle for the introduction and/or transfer of a base into a host cell in vitro, ex vivo, or in vivo, and may be a replicon that is bonded with another DNA fragment to bring about the replication of the bonded fragment, and “replication unit” refers to any genetic unit (e.g., a plasmid, a phage, a cosmid, a chromosome, or a virus), which functions as a self-unit of DNA replication in vivo, that is, is able to be replicated by self-regulation.

The vector according to the present invention may be linear DNA, plasmid DNA, or a recombinant viral vector, but the present invention is not limited thereto. Examples of the recombinant viral vector include a plasmid vector, a cosmid vector, a bacteriophage vector, and viral vectors such as an adenovirus vector, a lentivirus vector, a retrovirus vector, and an adeno-associated virus vector. Preferably, the vector is a retrovirus vector. The present inventors used pMXs-IRES-GFP retrovirus vector in a specific example.

The recombinant vector of the present invention preferably includes a transcription initiation factor to which RNA polymerase binds, such as a promoter, any operator sequence for regulating transcription, a sequence encoding an appropriate mRNA ribosome-binding site, sequences regulating the termination of transcription and translation, and a terminator, and more preferably, further includes a polyhistidine tag (an amino acid motif consisting of at least 5 histidine residues), a signal peptide gene, an endoplasmic reticulum retention signal peptide, a cloning site, or a marker gene for selection such as a tagging gene or an antibiotic-resistant gene for selecting a transformant. In the recombinant vector, the polynucleotide sequence of each gene is operably linked to a promoter. The term “operably linked” used herein refers to a functional linkage between a nucleotide expression regulatory sequence such as a promoter sequence and a different nucleotide sequence, and thus the regulatory sequence regulates the transcription and/or translation of the other nucleotide sequence.

The recombinant vector may be constructed using a prokaryotic cell or eukaryotic cell as a host. For example, when the vector of the present invention is an expression vector and a prokaryotic cell is a host, it generally includes a strong promoter capable of promoting transcription (e.g., a pLλ promoter, a trp promoter, a lac promoter, a tac promoter, or a T7 promoter), a ribosome binding site for the initiation of translation, and transcription/translation termination sequences. When a eukaryotic cell is a host, replication origins that make the vector operate in eukaryotic cells may include an fl replication origin, an SV40 replication origin, a pMBT replication origin, an adeno replication origin, an AAV replication origin, and a BBV replication origin, but the present invention is not limited thereto. In addition, a promoter derived from the genome of mammalian cells (e.g., a metallothionein promoter) or a promoter derived from a mammalian virus (e.g., an adenovirus late promoter, a vaccinia virus 7.5K promoter, an SV40 promoter, a cytomegalovirus promoter, and an HSV tk promoter) may be used, and as a transcription termination sequence, the recombinant vector generally has a polyadenylation sequence.

As the tagging gene, representatively, an Avi tag, a Calmodulin tag, a polyglutamate tag, an E tag, an FLAG tag, an HA tag, a His tag (polyhistidine tag), a Myc tag, an S tag, an SBP tag, an IgG-Fc tag, a CTB tag, a Softag 1 tag, a Softag 3 tag, a Strep tag, a TC tag, a V5 tag, a VSV tag, or an Xpress tag may be included. Preferably, the vector according to the present invention may include a myc tag. More preferably, the myc tag may include the base sequence of SEQ ID NO: 22.

In the present invention, the nucleic acid or the vector may be transduced or transfected to a virus production cell, that is, a packaging cell line. For “transduction” or “transfection,” various kinds of techniques generally used to introduce a foreign nucleic acid (DNA or RNA) into a prokaryotic or eukaryotic host cell, such as electrophoresis, calcium phosphate precipitation, DEAE-dextran transfection, or lipofection, may be used. A virus including a target gene (NK-CAR-coding nucleic acid molecule) according to the present invention may multiply in the packaging cell line and then may be released outside the cells, and the virus may be transduced to a target cell (i.e., a cell intended to ultimately express the NK-CAR of the present invention, such as an NK cell). The nucleic acid of the virus “transduced” into the cell is used to produce a target protein (NK cell-specific CAR) while inserted or not inserted into the genome of the cell.

The present invention may provide isolated cells into which the expression vector according to the present invention is introduced (transformed, transfected, or transduced). Here, the cells are cells for multiplying (amplifying) the expression vector, not cells for ultimately expressing NK-CAR. That is, the cells represent host cells in which the above-described nucleic acid molecule or expression vector is directly transduced/transformed/transfected. For example, when the expression vector is a viral vector, the cells may be packaging cells for producing viruses containing the viral vector. The selection of a suitable host is considered to be obvious to those of ordinary skill in the art from the content disclosed herein.

In addition, the present invention provides a method of preparing NK-CAR, which includes (S1) introducing the expression vector according to the present invention into cells; and (S2) culturing the expression vector-introduced cells.

Step (S1) is a step of amplifying the expression vector of the present invention, or introducing the expression vector into a host cell to express the vector. That is, in Step (S1), the expression vector may be introduced (transformed, transduced, or transfected) into an appropriate host cell to replicate the expression vector in the cell, to induce the expression of a protein from the expression vector, or (when using a viral vector) to induce the production of a virus containing the expression vector. Step (Si) may be performed appropriately by one of ordinary skill in the art depending on types of vectors and cells used herein. For example, when the expression vector is a viral vector, the expression vector may be introduced into a host cell through transduction.

Step (S2) is a step of culturing the cell to sufficiently amplify or express a target gene introduced into the cell therein. After introducing the expression vector into the host cell, the culturing may be performed for a sufficient period to allow the expression vector to express a target protein in the host cell (or a sufficient period to replicate the expression vector or a sufficient period to produce a virus containing the target gene). More preferably, the culturing may be performed for a sufficient period to release the protein into a culture medium for culturing the host cell (or a sufficient period for the virus containing the target gene to be released outside the cell).

The method may further include, after Step (S2), acquiring an NK-CAR produced (or amplified) from the cell, an expression vector including a nucleic acid molecule encoding the same, or a virus containing the expression vector. The acquisition method may be appropriately selected and adjusted by considering the characteristics of the CAR, expression vector, or virus produced in the host cell, the characteristics of the host cell, an expression method, or targeting of the CAR/expression vector/virus. If necessary, in order to release the CAR/expression vector/virus present in a specific organelle or cytoplasm in the cell outside the cell and then recover it, the cell may be lysed to the extent that it does not affect the functional structure of an antibody or an antigen-binding fragment thereof. In addition, the virus containing the expression vector can recover antibodies according to a method of acquiring the medium in which the host cell is cultured and removing impurities by centrifugation.

The acquired CAR/expression vector/virus may be further subjected to more removal of impurities through chromatography, filtration using a filter, or dialysis, and concentration. The isolation or purification of the acquired CAR may be performed by a conventional isolation and purification method that is used on a protein, for example, chromatography. Examples of the chromatography may include affinity chromatography, ion exchange chromatography, or hydrophobic chromatography, including a protein A column, a protein G column, or a protein L column. In addition to the above chromatography, the CAR may be isolated and purified by additionally combining filtration, ultrafiltration, salting out, and dialysis. Virus isolation or purification may be performed by filtering the medium through a porous filter to remove impurities, and concentrating the resulting filtrate.

In addition, the present invention provides a method of preparing NK cells expressing NK-CAR, which includes introducing an expression vector including a nucleic acid molecule encoding the NK-CAR according to the present invention into NK cells. That is, the present invention provides a method of preparing CAR-NK cells, which includes the CAR according to the present invention. The expression vector may be an expression vector amplified by the above-described method of preparing the NK-CAR. The expression vector may be introduced alone into NK cells, or introduced in a form contained in a virus. One of ordinary skill in the art may select an appropriate introduction method depending on types of expression vectors and NK cells. For example, when the expression vector is a viral vector, the NK cells may be infected with the virus containing the expression vector, and thus the vector may be introduced into the cells (transduction). The nucleic acid of the virus transduced into the cells may be inserted into the genome of the NK cell, or may be expressed without being inserted, and therefore the NK-CAR may be expressed.

In addition, the present invention provides a pharmaceutical composition for preventing or treating cancer, which includes NK cells expressing the NK-CAR according to the present invention; an expression vector including a nucleic acid molecule encoding the NK-CAR; and/or cells including the expression vector as active ingredients. Here, the antigen-binding domain of the NK-CAR is an antigen-binding domain specifically binding to a tumor antigen. The NK cells may be autologous cells or allogeneic cells of an individual subject to administration of the composition.

In the present invention, “cancer” is used with the same meaning as “tumor,” and refers to a condition typically characterized by uncontrolled cell growth or proliferation. Types of cancer that can be prevented or treated using the CAR according to the present invention include solid tumors and non-solid tumors (e.g., blood cancer). In addition, in the present invention, types of cancer may include, but are not limited to, carcinomas, blastomas, sarcomas, specific leukemic or lymphoid malignant tumors, and benign and malignant tumors, such as sarcomas, carcinomas, and melanomas.

Specific types of cancer of the present invention are not limited, and may be selected from colorectal cancer, rectal cancer, colon cancer, thyroid cancer, oral cancer, pharynx cancer, larynx cancer, cervical cancer, brain cancer, lung cancer, ovarian cancer, bladder cancer, kidney cancer, liver cancer, pancreatic cancer, prostate cancer, skin cancer, tongue cancer, breast cancer, uterine cancer, stomach cancer, bone cancer, lymphoma, blood cancer, squamous cell carcinoma, adenocarcinoma of the lung, peritoneal cancer, skin melanoma, ocular melanoma, perianal cancer, esophageal cancer, small intestine cancer, endocrine cancer, parathyroid cancer, adrenal cancer, soft tissue sarcoma, urethral cancer, gastrointestinal cancer, glioblastoma, endometrial cancer, salivary gland cancer, vulvar cancer, and head and neck cancer. Preferably, the cancer according to the present invention is cancer that expresses a target antigen recognized by the CAR of the present invention. That is, the cancer is cancer expressing a tumor antigen that can be recognized by an antigen-binding domain of the CAR of the present invention. For example, when the CAR according to the present invention includes anti-CD19 scFv as a cancer cell-specific antigen-binding domain, the cancer is preferably cancer in which CD19 is overexpressed or has higher activity in cancer cells compared to normal cells.

In the present invention, “blood cancer” refers to cancer that occurs in the components of blood (white blood cells, red blood cells, platelets, etc.), the bone marrow that makes blood, or the lymphatic system that constitutes the immune system (lymphocytes, lymph nodes, lymph vessels, etc.). Representative examples of blood cancer include leukemia, malignant lymphoma, and multiple myeloma. More specific examples of blood cancer include acute myeloid leukemia, acute lymphoblastic leukemia, chronic myelogenous leukemia, multiple myeloma, and lymphoma.

Preferably, the cancer is selected from lymphoma, B cell lymphoma, acute lymphoma, bucket lymphoma, diffuse large B cell lymphoma, follicular lymphoma, MALT lymphoma, marginal zone lymphoma, peripheral T cell lymphoma, anaplastic large cell lymphoma, lymphoblastic lymphoma, and Hodgkin's lymphoma.

The content of NK cells, an expression vector, and/or cells including an expression vector which expresses the NK-CAR in the composition of the present invention can be appropriately adjusted according to symptoms of a disease, the degree of the progression of symptoms, and a patient's condition, and is, for example, 0.0001 to 99.9 wt % or 0.001 to 50 wt % based on the total weight of the composition, but the present invention is not limited thereto. The content ratio is a value based on the dry weight from which a solvent is removed.

The pharmaceutical composition according to the present invention may further include an appropriate carrier, excipient, and diluent, which are conventionally used in the preparation of a pharmaceutical composition. The excipient may be, for example, one or more selected from the group consisting of a diluent, a binder, a disintegrant, a lubricant, an adsorbent, a humectant, a film-coating material, and a controlled-release additive.

The pharmaceutical composition according to the present invention may be formulated in the form of a powder, a granule, a sustained-release granule, an enteric granule, a liquid, an ophthalmic solution, an elixir, an emulsion, a suspension, a spirit, a troche, aromatic water, a lemonade, a tablet, a sustained-release tablet, an enteric tablet, a sublingual tablet, a hard capsule, a soft capsule, a sustained-release capsule, an enteric capsule, a pill, a tincture, a soft extract, a dry extract, a fluid extract, an injection, a capsule, a perfusate, a plaster, a lotion, a paste, a spray, an inhalant, a patch, a sterile injection, or an external preparation such as an aerosol according to a conventional method, and the external preparation may be formulated in a cream, a gel, a patch, a spray, an ointment, a plaster, a lotion, a liniment, a paste or a cataplasma.

Carriers, excipients, and diluents that can be included in the pharmaceutical composition according to the present invention may include lactose, dextrose, sucrose, an oligosaccharide, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propylhydroxybenzoate, talc, magnesium stearate, and mineral oil.

The composition according to the present invention may be prepared using a diluent or an excipient such as a filler, a thickening agent, a binder, a wetting agent, a disintegrant, and a surfactant, which are commonly used.

Additives for a tablet, powder, granule, capsule, pill and troche may include excipients such as corn starch, potato starch, wheat starch, lactose, sucrose, glucose, fructose, di-mannitol, precipitated calcium carbonate, synthetic aluminum silicate, calcium monohydrogen phosphate, calcium sulfate, sodium chloride, sodium bicarbonate, purified lanolin, microcrystalline cellulose, dextrin, sodium alginate, methylcellulose, carboxymethylcellulose, sodium carboxymethylcellulose, kaolin, urea, colloidal silica gel, hydroxypropyl starch, hydroxypropyl methyl cellulose (HPMC) 1928, HPMC 2208, HPMC 2906, HPMC 2910, propylene glycol, casein, calcium lactate and Primojel; binders such as gelatin, gum arabic, ethanol, agar powder, cellulose acetate phthalate, carboxymethyl cellulose, carboxymethyl cellulose calcium, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch powder, hydroxypropylcellulose, hydroxypropylmethylcellulose, polyvinyl alcohol and polyvinylpyrrolidone; disintegrants such as hydroxypropylmethylcellulose, corn starch, agar powder, methylcellulose, bentonite, hydroxypropyl starch, sodium carboxymethylcellulose, calcium citrate, sodium lauryl sulfate, silicic anhydride, 1-hydroxypropyl cellulose, dextran, an ion exchange resin, polyvinyl acetate, formaldehyde-treated casein and gelatin, alginic acid, amylose, guar gum, sodium bicarbonate, polyvinylpyrrolidone, calcium phosphate, gelled starch, amylopectin, pectin, sodium polyphosphate, ethyl cellulose, sucrose, magnesium aluminum silicate, a di-sorbitol solution and light anhydrous silicic acid; and lubricants such as calcium stearate, magnesium stearate, stearic acid, hydrogenated vegetable oil, talc, lycopodium powder, kaolin, petrolatum, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, a higher fatty acid, a higher alcohol, silicone oil, paraffin oil, polyethylene glycol fatty acid ether, starch, sodium chloride, sodium acetate, sodium oleate, dl-leucine and light anhydrous silicic acid.

Additives for a liquid according to the present invention may be water, diluted hydrochloric acid, diluted sulfuric acid, sodium citrate, monostearate sucrose, polyoxyethylene sorbitol fatty acid esters (Tween esters), polyoxyethylene monoalkylethers, lanolin ethers, lanolin esters, acetic acid, hydrochloric acid, aqueous ammonia, ammonium carbonate, potassium hydroxide, sodium hydroxide, prolamine, polyvinylpyrrolidone, ethyl cellulose, and sodium carboxymethylcellulose.

A syrup according to the present invention may include a solution of white sugar, a different type of sugar, or a sweetener, and if necessary, a fragrance, a colorant, a preservative, a stabilizer, a suspending agent, an emulsifier, or a thickener.

An emulsion according to the present invention may include purified water, and if necessary, an emulsifier, a preservative, a stabilizer, or a fragment.

A suspension according to the present invention may include a suspending agent such as acacia, tragacanth, methylcellulose, carboxymethylcellulose, sodium carboxymethylcellulose, microcrystalline cellulose, sodium alginate, hydroxypropylmethyl cellulose (HPMC), HPMC 1828, HPMC 2906, or HPMC 2910, and if necessary, a surfactant, a preservative, a stabilizer, a colorant, or a fragrance.

An injection according to the present invention may include a solvent such as injectable sterile water, 0.9% sodium chloride for injection, Ringer's solution, dextrose for injection, dextrose+sodium chloride for injection, PEG, lactated Ringer's solution, ethanol, propylene glycol, non-volatile oil-sesame oil, cottonseed oil, peanut oil, soybean oil, corn oil, ethyl oleate, isopropyl myristic acid or benzene benzoate; a solubilizing agent such as sodium benzoate, sodium salicylate, sodium acetate, urea, urethane, monoethylacetamine, butazolidine, propylene glycol, Tween, nicotinamide, hexamine or dimethylacetamide; a buffer such as a weak acid and a salt thereof (acetic acid and sodium acetate), a weak base and a salt thereof (ammonia and ammonium acetate), an organic compound, a protein, albumin, peptone, or gums; an isotonic agent such as sodium chloride; a stabilizer such as sodium bisulfite (NaHSO₃), carbon dioxide gas, sodium metabisulfite (Na₂S₂O₃), sodium sulfite (Na₂SO₃), nitrogen gas (N₂) or ethylenediaminetetracetic acid; an antioxidant such as sodium bisulfide 0.1%, sodium formaldehyde sulfoxylate, thiourea, disodium ethylenediaminetetraacetate or acetone sodium bisulfite; an analgesic such as benzyl alcohol, chlorobutanol, procaine hydrochloride, glucose or calcium gluconate; or a suspending agent such as sodium CMC, sodium alginate, Tween 80 or aluminum monostearate.

A suppository according to the present invention may include a base such as cacao butter, lanolin, Witepsol, polyethylene glycol, glycerogelatin, methyl cellulose, carboxymethylcellulose, a mixture of stearate and oleate, Subanal, cottonseed oil, peanut oil, palm oil, cacao buffer+ cholesterol, lecithin, Lanette wax, glycerol monostearate, Tween or Span, Imhausen, monolene (propylene glycol monostearate), glycerin, Adeps solidus, Buytyrum Tego-G, Cebes Pharma 16, hexalide base 95, Cotomar, Hydrokote SP, S-70-XXA, S-70-XX75 (S-70-XX95), Hydrokote 25, Hydrokote 711, Idropostal, Massa estrarium, (A, AS, B, C, D, E, I, T), Mass-MF, Masupol, Masupol-15, neosuppostal-N, paramount-B, supposiro (OSI, OSIX, A, B, C, D, H, L), suppository base IV types (AB, B, A, BC, BBG, E, BGF, C, D, 299), Suppostal (N, Es), Wecoby (W, R, S, M, Fs), or a Tegester triglyceride base (TG-95, MA, 57).

A solid formulation for oral administration may be a tablet, a pill, a powder, a granule or a capsule prepared by mixing at least one or more excipients, for example, starch, calcium carbonate, sucrose, lactose and gelatin. In addition to the simple excipients, lubricants such as magnesium stearate and talc may also be used.

A liquid formulation for oral administration may be a suspension, a liquid for internal use, an emulsion or a syrup, and a generally used simple diluent such as water or liquid paraffin, as well as various types of excipients, for example, a wetting agent, a sweetener, a fragrance and a preservative may be included. A formulation for parenteral administration may be a sterilized aqueous solution, a non-aqueous solvent, a suspension, an emulsion, a lyophilizing agent or a suppository. As the non-aqueous solvent or suspension, propylene glycol, polyethylene glycol, a vegetable oil such as olive oil, or an injectable ester such as ethyl oleate may be used.

The pharmaceutical composition of the present invention may be administered at a pharmaceutically effective amount. The term “pharmaceutically effective amount” used herein refers to an amount sufficient for treating a disease at a reasonable benefit/risk ratio applicable for medical treatment, and an effective dosage may be determined according to parameters including a type of a patient's disease, severity, drug activity, sensitivity to a drug, administration time, an administration route and an excretion rate, the duration of treatment and drugs simultaneously used, and other parameters well known in the medical field.

The pharmaceutical composition of the present invention may be administered separately or in combination with other therapeutic agents, and may be sequentially or simultaneously administered with a conventional therapeutic agent, or administered in a single or multiple dose(s). In consideration of all of the above-mentioned parameters, it is important to achieve the maximum effect with the minimum dose without side effects, and such a dose may be easily determined by one of ordinary skill in the art.

The pharmaceutical composition of the present invention may be administered into a subject via various routes. All administration routes may be considered, and the pharmaceutical composition of the present invention may be administered by, for example, oral administration, subcutaneous injection, intraperitoneal administration, intravenous, intramuscular or intrathecal injection, sublingual administration, buccal administration, rectal insertion, vaginal insertion, ocular administration, ear administration, nasal administration, inhalation, spraying through the mouth or nose, skin administration, or transdermal administration.

The pharmaceutical composition of the present invention is determined according to the type of drug as an active ingredient as well as various related parameters such as a disease to be treated, an administration route, a patient's age, sex, and body weight, and the severity of a disease.

In the present invention, “individual” refers to a target subject in need of treatment for a disease, and more specifically, a mammal such as a human or a non-human primate, a mouse, a rat, a dog, a cat, a horse, or a cow.

In the present invention, “administration” refers to providing the given composition of the present invention to a subject by any suitable method.

In the present invention, “prevention” refers to all actions involved in inhibiting or delaying the onset of a desired disease, “treatment” refers to all actions involved in improving or beneficially changing a desired disease and its metabolic abnormalities by administering the pharmaceutical composition according to the present invention, and “alleviation” refers to all actions involved in reducing parameters related to a desired disease, e.g., the severity of symptoms, by administering the composition according to the present invention.

In addition, the present invention provides a kit for preventing or treating cancer, which includes NK cells expressing the NK-CAR according to the present invention; an expression vector including a nucleic acid molecule encoding the NK-CAR; and/or including the expression vector. The kit according to the present invention is not limited to the specific form as long as it is for preventing or treating cancer, and may include any components and devices for the preparation and storage of the CAR according to the present invention, the introduction and expression into NK cells, and the administration of the NK cells without limitation.

In addition, the present invention provides a cellular therapeutic agent for preventing or treating cancer, which includes NK cells that express NK-CAR according to the present invention as an active ingredient.

In the present invention, “cellular therapeutic agent” refers to cells or tissue prepared through isolation, culture, and special manipulation from humans, and used for the purpose of diagnosis, prevention, or treatment of a specific disease, is defined as a medication by the US FDA. Specifically, cellular therapeutic agents are prepared through a series of processes of amplifying and selecting live autologous, allogeneic, or heterologous cells ex vivo, or changing the biological characteristics of cells through other methods to restore a cell or tissue function, and then used in diagnosis, prevention, and treatment of a disease.

Hereinafter, preferred examples will be presented to help in understanding the present invention. However, the following examples are merely provided to better explain the present invention, and the content of the present invention is not limited to the following examples.

### EXAMPLES

### Example 1. Preparation of Anti-CD19-CAR Expression Vector

Based on an NK cell-specific signaling domain, three types of artificial synthetic genes of anti-CD19-DAP10-2B4-CD3ζ CARs according to the present invention were produced (see (1) to (3) below), and as a control, an artificial synthetic gene of anti-CD19-CD28-CD3ζ CAR was also produced (see (4) below).

The type and configuration of each CAR are as follows:
(1) a single molecule of chimeric protein (named “NK-CAR1”) consisting of a myc-tag, a scFv of anti-CD19 monoclonal antibody that binds to CD19, a CD8 hinge domain, a DAP10 extracellular domain, a DAP10 transmembrane domain, a DAP10 cytoplasmic domain, a 2B4 cytoplasmic domain, and a CD3ζ (also referred to as CD3z) cytoplasmic domain;
(2) a single molecule of chimeric protein (named “NK-CAR2”) consisting of a myc-tag, a scFv of anti-CD19 monoclonal antibody that binds to CD19, a CD28 hinge domain, a DAP10 extracellular domain, a DAP10 transmembrane domain, a DAP10 cytoplasmic domain, a 2B4 cytoplasmic domain, and a CD3ζ cytoplasmic domain;
(3) a single molecule of chimeric protein (named “NK-CAR3”) consisting of a myc-tag, a scFv of anti-CD19 monoclonal antibody that binds to CD19, a CD8 hinge domain, a CD28 transmembrane domain, a DAP10 cytoplasmic domain, a 2B4 cytoplasmic domain, and a CD3ζ cytoplasmic domain; and
(4) a single molecule of chimeric protein (T cell-derived CAR; named “2^{nd} CAR” or “2^{nd} generation CAR”) consisting of a myc-tag, a scFv of anti-CD19 monoclonal antibody that binds to CD19, a CD8 hinge domain, a CD28 transmembrane domain, a CD28 cytoplasmic domain, and a CD3ζ cytoplasmic domain.

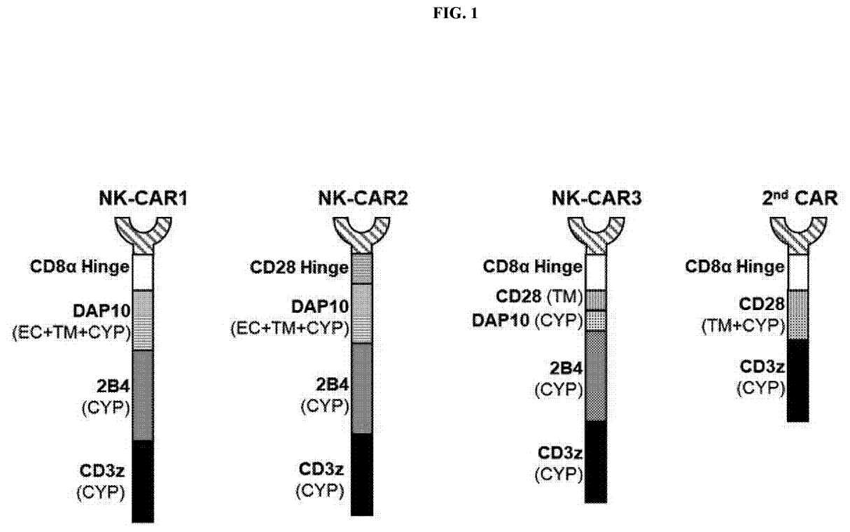

Three types of pMXs-CAR-IRES-GFP retrovirus vectors expressing the NK-CAR according to the present invention and one type of T-CAR-expressing retrovirus vector were produced as a control by cloning a pMXs-IRES-GFP retrovirus vector in which a nucleic acid fragment containing each of the artificial synthetic genes was digested with EcoR1-Xho1. That is, the vector encodes the CAR according to the present invention with a fluorescent protein GFP. The structures of the NK-CART, NK-CAR2, and NK-CAR3 according to the present invention, and the control 2^{nd} CAR are shown in FIG. 1.

### Example 2. Preparation of NK-CAR Retrovirus Solution

E. coli DH5α was transformed with each of the four types of pMXs-CAR-IRES-GFP retrovirus vectors produced in Example 1, thereby obtaining transformants. Plasmid DNA replicated in each transformant was purified using a HiSpeed Plasmid Midi Kit (Qiagen, #12643), and prepared as DNA for transfection (transduction). The DNA for transfection was applied to manipulation as below.

Using X-tremeGENE9 (Roche, #6365787001) according to the manufacturer's instructions, the DNA for transfection was transduced into a retrovirus packaging cell line, Plat A cells. 24 hours after transduction, a medium was exchanged, and 24 hours after medium exchange, a supernatant containing the amphotropic retrovirus was obtained and filtered through a 0.45 μm filter (Nalgene, #725-2545). Using the supernatant, the amphotropic viruses were subjected to spinfection with 10 μg/mL polybrene (Sigma, #H9268) twice into an NK cell line NKL and an NK92 cell line under conditions including 700 G, 30 minutes, and 32° C. according to the manufacturer's instructions.

Among the cells on day 3 of viral infection, GFP-expressing cells were isolated using a BD FACSAria™ II Cell Sorter (BD Biosciences). The cells on day 3 of isolation were stained with Alexa Fluor™ 647-Myc-Tag (9B11) Mouse mAb (Cell Signaling, #2233S), and assessed using BD Accuri™C6 (BD Biosciences). GFP-positive cells are cells infected by the retrovirus produced in this example, and Alexa Fluor™ 647-positive cells are cells expressing the NK-CAR according to the present invention.

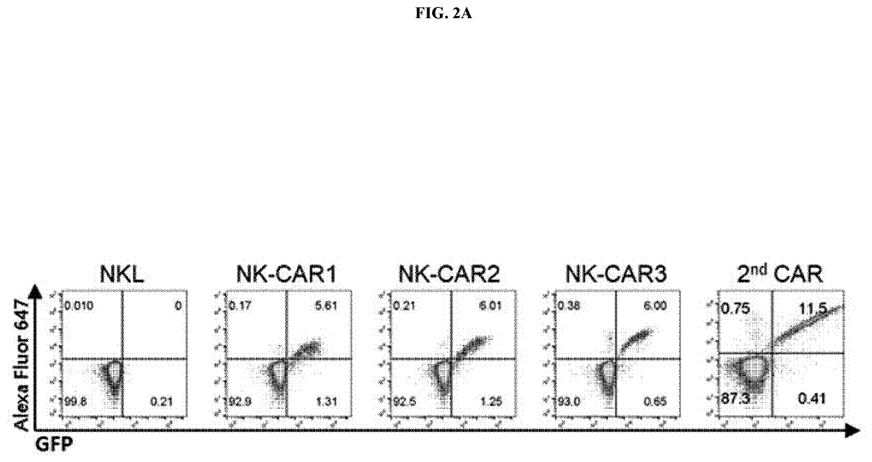

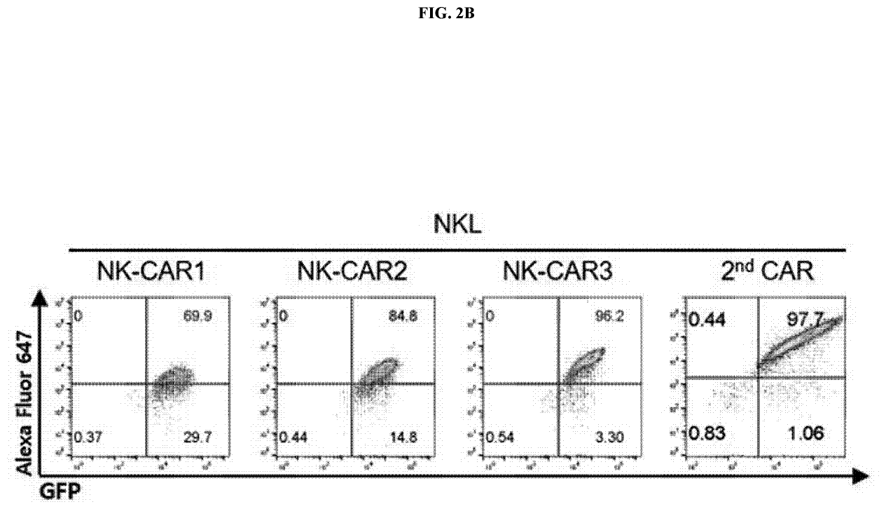

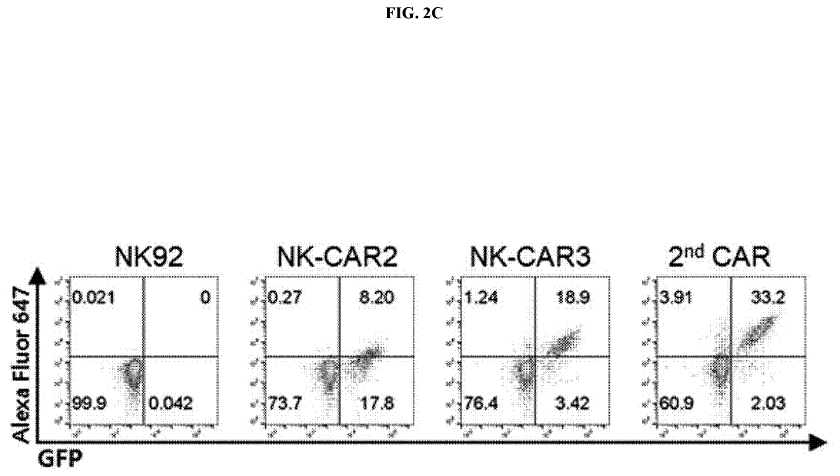

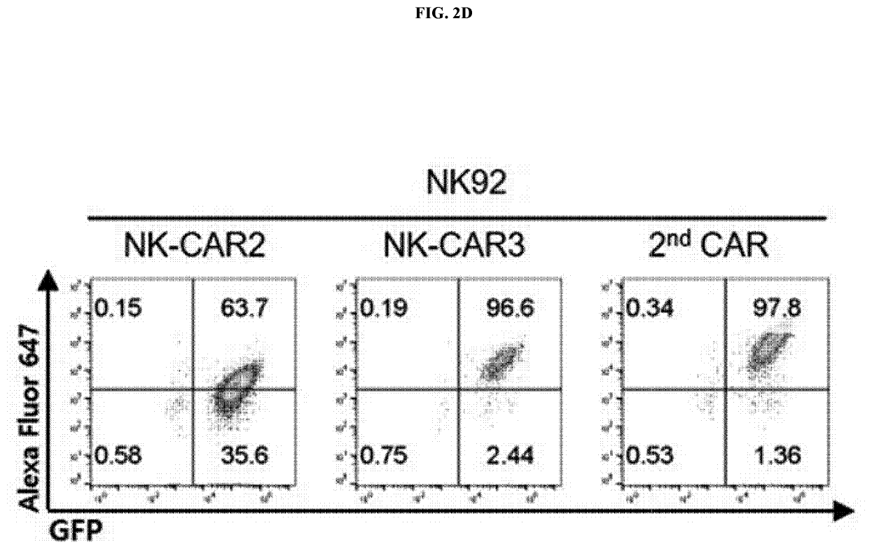

First, among the GFP-positive cells (NKL cells infected by the virus), the proportion of the Alexa Fluor™ 647-positive cells, that is, the proportion of the NLK cells expressing the NK-CAR, was measured. As shown in FIG. 2A, the proportion of the NKL cells infected by the CAR-retrovirus was approximately 7%, and among them, the proportion of the NKL cells expressing the NK-CAR according to the present invention was approximately 85%. The proportion of the NKL cells infected by the 2^{nd} CAR virus was approximately 12%, and among them, the proportion of the NKL cells expressing 2^{nd} CAR was detected at approximately 95%.

Subsequently, among the NKL cells on day 3 of virus infection, the NKL cells expressing GFP were isolated using a BD FACSAria™ II Cell Sorter (BD Biosciences) and cultured, and to confirm GFP and NK-CAR expression, as described above, the cultured cells were stained with Alexa Fluor™ 647-Myc-Tag (9B11) mouse mAb and then assessed by BD Accuri™C6 (BD Biosciences). As a result, GFP was expressed in 99% or more of the cells, confirming that only the virus-infected NKL cells were isolated and cultured (FIG. 2B). As a result of observing the proportion of NK-CAR-expressing cells with respect to the GFP-positive cells, it was confirmed that the proportion of the NK-CAR1-expressing cells was 70%, the proportion of the NK-CAR2-expressing cells was 85%, and the proportion of the NK-CAR3-expressing cells was 96% (FIG. 2B). The above results reveal that the structural difference of NK-CARs affects protein expression.

In addition, among the GFP-positive cells (virus-infected NK92 cells), the proportion of the Alexa Fluor® 647-positive cells and the proportion of NK-CAR-expressing NK92 cells were measured. As shown in FIG. 2C, the proportion of the NK92 cells injected by the NK-CAR virus was approximately 22 to 26%, and among them, the proportion of the NK-CAR-expressing NK92 cells was approximately 32 to 85%. It was confirmed that the proportion of the NK92 cells infected by the 2^{nd} CAR virus was approximately 35%, and among them, the proportion of the 2^{nd} CAR-expressing NK92 cells was approximately 94%.

Subsequently, among the NK92 cells on day 3 of virus infection, the GFP-expressing NK 92 cells were isolated using a BD FACSAria™ II Cell Sorter (BD Biosciences) and cultured, and to confirm GFP and NK-CAR expression, as described above, the cultured cells were stained with Alexa Fluor™ 647-Myc-Tag (9B11) mouse mAb and then assessed by BD Accuri™C6 (BD Biosciences). The result showed that GFP was expressed in 99% of the cells, confirming that only virus-infected NK 92 cells were isolated and cultured (FIG. 2D). As a result of observing the proportion of NK-CAR-expressing cells with respect to the GFP-positive cells, it was confirmed that the proportion of the NK-CAR2-expressing cells was 63.7%, and the proportion of the NK-CAR3-expressing cells was 96.6% (FIG. 2D). The above results reveal that the structural difference of NK-CARs affects protein expression.

### Example 3. Evaluation of Antigen-Specific Cytolytic Activity of NK-CAR-Expressing NK Cells

In this example, the cytolytic activity of NK cells into which NK-CAR was introduced, produced by the above example, was evaluated. First, to evaluate the cytolytic activity of the NK-CAR-introduced NKL cells, a CD19-positive cell line REH on day 2 of culture was used as target cells, and NK-CAR-expressing NKL cells on day 3 of culture, which had rested for 24 hours, and as a control, NKL cells into which pMXs-IRES-GFP (empty vector) was introduced, were used as effector cells. To allow the effector cells to rest, the cells on day 2 of culture were washed with serum-free RPMI1640, suspended in 10 mL of a rested RPMI1640 (+5% FBS, 0.5% pen/strep, 0.5% sodium pyruvate) medium and then incubated for 24 hours. In addition, to evaluate the cytolytic activity of the NK-CAR-introduced NK92 cells, a CD19-positive cell line REH and Ramos on day 2 of culture were used as target cells, and NK-CAR-expressing NK92 cells on day 2 of culture, which had not rested, and NK92 into which pMXs-IRES-GFP (empty vector) was introduced as a control were used as effector cells.

The cytolytic activity of the NK-CAR-expressing NKL and NK92 cells was measured through a europium assay. First, target cells were suspended in rested IMDM (+5% FBS, 0.5% pen/strep) to be 1.0×10⁶ cells/400 μL, 40 μM BATDA (Perkin Elmer, #C136-100) was added and kept warm for 30 minutes in a CO₂ incubator, and equilibrated with 5.0% CO₂ gas at 37° C. Afterward, the resulting cells were washed with 1 mM sulfinpyrazone (Sigma, #S9509)-containing rested IMDM (1 mM sulfinpyrazone-containing rested IMDM was named an assay medium), and suspended with the assay medium by 100 μL per well to be 5.0×10⁴ cells/1 mL. In addition, the effector cells were suspended in the assay medium, and added at 100 μL such that the effector to target (E:T) ratio became 20:1, 10:1, or 5:1 for the NK-CAR-expressing NKL cells, and 4:1, 2:1, or 1:1 for the NK-CAR-expressing NK92 cells. Instead of the effector cells, a medium was prepared as a spontaneous control, and a well to which 100 μL of 2% Triton X-100 was added as a maximum lysis control.

After preparing the cells and each control, a 96-well plate was kept warm in a CO₂ incubator equilibrated with 5.0% CO₂ gas at 37° C. The NK-CAR-expressing NKL cells warmed for 2 hours, and the NK-CAR-expressing NK92 cells warmed for 30 minutes.

Subsequently, 20 μL of a supernatant was reacted in a 20% europium solution (Perkin Elmer, #C135-100) containing 0.3 M acetic acid for 5 minutes, and fluorescence intensity was measured using a Victor X4 multilabel plate reader (Perkin Elmer) to calculated cytolytic activity (%). The calculation formula of the cytolytic activity (%) is as follows: Cytolytic activity (%)=100×(measured value of each well-measured value of spontaneous control)/(measured value of maximum lysis control-measured value of spontaneous control).

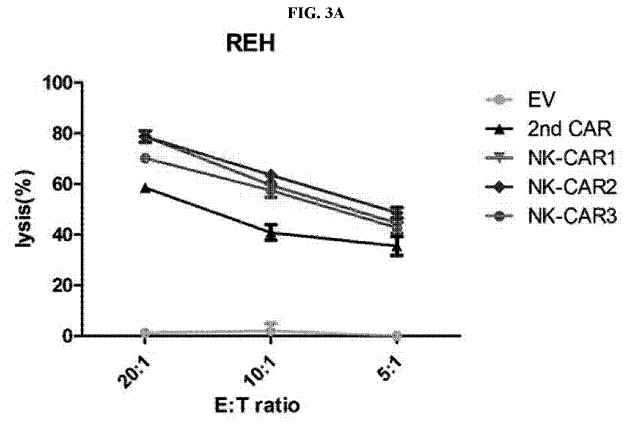

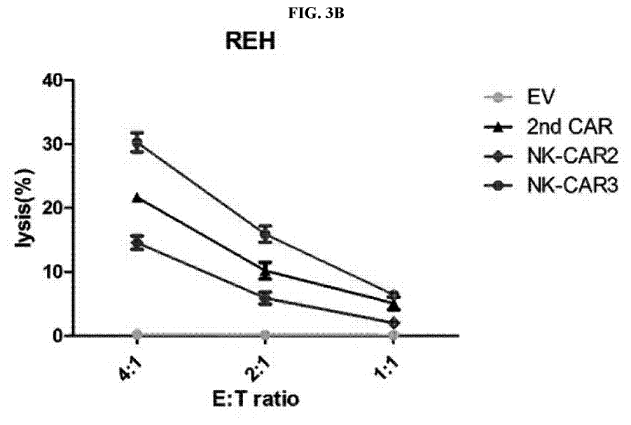

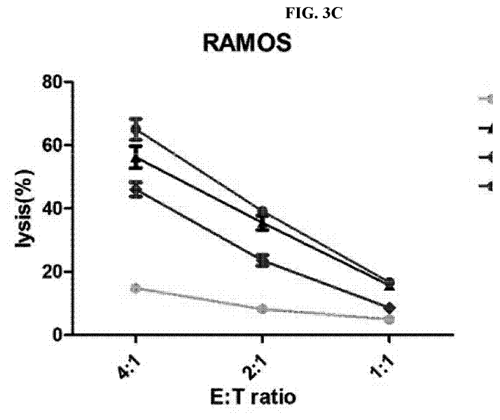

The results of the europium assay are shown in FIGS. 3A to 3C. First, FIG. 3A shows the cytolytic activity of NK-CAR-introduced NKL cells. An empty vector (EV) is NKL cells infected by a virus made by a pMXs-IRES-GFP vector, and 2^{nd} CAR and NK-CAR are NKL cells infected by a virus made by a pMXs-CAR-IRES-GFP vector. As shown in FIG. 3A, when the effector-to-target ratio was 20:1, the EV group did not kill the CD19-positive cell line REH, but the 2^{nd} CAR killed approximately 60% REH cells, and the NK-CAR killed approximately 70 to 80% REH cells. In addition, even when the effector-to-target ratio was 10:1 or 5:1, it was shown that the REH cytolytic activity of the NK-CAR-expressing NKL cells according to the present invention was higher than the control, 2^{nd} CAR-expressing cells.

FIGS. 3B and 3C show the cytolytic activities of NK-CAR-introduced NK92 cells to REH cells (FIG. 3B) and RAMOS cells (FIG. 3C). An empty vector (EV) is cells infected by a virus made by a pMXs-IRES-GFP vector, and 2^{nd} CAR and NK-CAR are NK92 cells infected by a virus made by a pMXs-CAR-IRES-GFP vector. As shown in FIGS. 3B and 3C, in both the REH cells and the RAMOS cells, compared to the 2^{nd} CAR-expressing cells, the cytolytic activity of the NK-CAR-expressing cells of the present invention was significantly higher. These results are the same at all effector-to-target ratios.

These results show that, as NK-CAR was introduced into NKL and NK92 cells, the NKL and NK92 cells acquired the ability to recognize CD19 and kill CD19-specific target cells. In addition, it shows that the NK-CAR3 of the present invention produced based on the specific NK activation receptor combination (NKG2D+2B4) is particularly effective on NK cell activation. Particularly, the NK-CAR3-expressing cells showed a higher cytolytic activity than other NK-CAR-expressing cells, demonstrating that, among the NK-CARs according to the present invention, NK-CAR3 is particularly effective on NK cell activation.

### Example 4. Confirmation of Cytokine Secretion Ability of NK-CAR-Expressing NK Cells

Macrophage inflammatory protein-1α (MIP-1α; also called CCL3) is a chemotactic cytokine known as a chemokine secreted from activated NK cells. Granzyme B is a serine protease most commonly found in granules ofNK cells and cytotoxic T cells, and is an important factor in inducing a perforin-dependent target cell death mechanism by the cells, secreted together with perforin. That is, MIP-1α and granzyme B become measures of the degree of NK cell activation, and to confirm whether the NK-CAR according to the present invention can effectively induce the cytolytic function of the NKL cells, MIP-la and granzyme B levels of the NK-CAR-expressing cells were measured.

A CD19-positive cell line REH on day 2 of culture was used as target cells, and NK-CAR-expressing NKL cells on day 3 of culture, which had rested for 24 hours, 2^{nd} CAR-introduced NKL cells, pMXs-IRES-GFP (EV)-introduced NKL cells, and simple NKL cells (NKL cells into which a vector was not introduced) were used as effector cells as a control. To allow the effector cells to rest, the cells on day 2 of culture were washed with serum-free RPMI1640, suspended in 10 mL of rested RPMI1640 (+5% FBS, 0.5% pen/strep, 0.5% sodium pyruvate), and then cultured for 24 hours.

The target cells REH were suspended in rested IMDM (+5% FBS, 0.5% pen/strep) to be 1.0×10⁶ cells/100 μL/sample, and the effector cells were suspended to be 0.5×10⁶ cells/150 μL/sample, and then seeded in a 96-well plate.

These cells were co-cultured in a CO₂ incubator equilibrated with 5.0% CO₂ gas at 37° C. for 8 hours. Afterward, a supernatant was acquired by centrifuging the cell culture, and the MIP-1α and granzyme B secretion levels in the supernatant were confirmed using a human CCL3/MIP-1alpha DuoSet ELISA kit (R&D Systems, #DY270) and a human Granzyme B DuoSet ELISA kit (R&D Systems, #DY2906) according to the manufacturer's instructions.

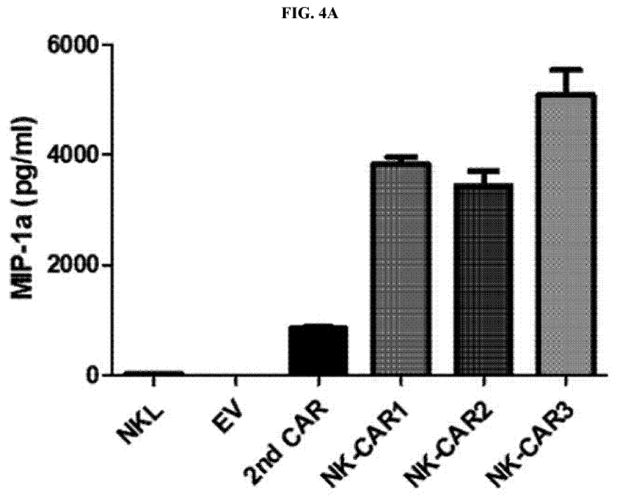

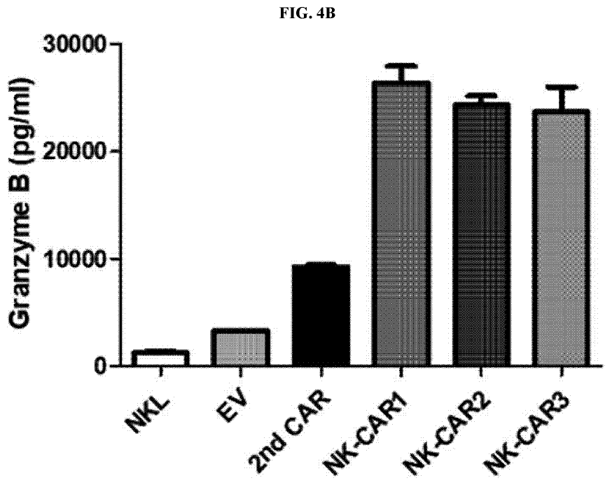

As a result, as shown in FIGS. 4A and 4B, MIP-lu and granzyme B, secreted by activated NK cells, were shown to be produced and secreted only by CAR-expressing cells. In addition, the cytokine secretion levels of the NK-CAR-expressing NKL cells according to the present invention were shown to be significantly higher than those of the 2^{nd} CAR-expressing NKL cells. The result shows that the NK-CAR according to the present invention, based on the specific receptor combination, effectively activates the target cell killing function of NK cells and promotes cytokine secretion.

### Example 5. Confirmation of Activation of AKT and ERK Signaling Pathway in NK-CAR-Expressing NK Cells

NK cells recognizing a target antigen activate AKT and ERK signals to exhibit an antitumor effect. Accordingly, it was assessed whether the NK-CAR-expressing NK cells according to the present invention effectively recognized a target antigen (CD19), causing specific AKT and ERK signal activation. To this end, the CD19-positive cell line REH or recombinant human CD19 Fc chimera was used as a stimulant, and NK-CAR-introduced NKL cells on day 3 of culture, which had rested for 24 hours, and the control, NKL cells, were stimulated, assessing the changes in AKT and ERK signals. To allow the cells to rest, the cells on day 2 of culture were washed with serum-free RPMI1640, suspended in 10 mL of rested RPMI1640 (+5% FBS, 0.5% pen/strep, 0.5% sodium pyruvate), and then cultured for 24 hours.

The process of stimulating NKL cells with REH cells and acquiring cell lysates is as follows. Each of the CD19-positive cell line, REH cells, anti CD19-DAP10-2B4 CAR (the structure of NK-CAR2 without a CD3ζ cytoplasmic domain)-introduced NKL cells, and the control NKL cells was suspended in rested RPMI (+5% FBS, 0.5% pen/strep, 0.5% sodium pyruvate) to be 5.0×10⁶ cells/100 μL/sample, and kept cool at 4° C. for 10 minutes. Subsequently, REH cells and CAR-introduced NKL cells; and REH cells and the control NKL cells were mixed and centrifuged, and stored cool in a pellet form at 4° C. for 10 minutes. Afterward, stimulation was performed by putting the pellet in a water bath at 37° C., stimulating it for 2 minutes and 5 minutes, and after stimulation, cooling it on ice for 5 minutes to terminate the stimulation. Afterward, the cells were washed with DPBS, and vortexed by adding a lysis buffer, followed by lysis on ice. Afterward, the resulting product was centrifuged, thereby obtaining a cell lysate.

The process of obtaining a cell lysate by stimulating the NKL cells with recombinant human CD19 Fc chimera is as follows. 4.0×10⁷ beads/sample of protein G (Invitrogen, #1003D) was washed with a wash buffer (1×PBS+0.01% Tween-20+1% FBS). Here, 4 μg/sample of the recombinant human CD19 Fc chimera (R&D, #9269-CD) was added, and rotation-incubation was performed at 4° C. for 1 hour. Afterward, the incubated cells were washed with a wash buffer, and suspended in rested RPMI (+5% FBS, 0.5% pen/strep, 0.5% sodium pyruvate) to be 4.0×10⁷ beads/100 μL/sample, thereby producing a recombinant human CD19 Fc chimera-coated protein G beads solution. 2.0×10⁶ cells/sample each of the anti-CD19-DAP10-2B4 CAR (the structure of NK-CAR2 without a CD3ζ cytoplasmic domain)-introduced NKL cells and the control NKL cells were centrifuged to prepare pellets, and then cooled at 4° C. for 10 minutes. The cells were resuspended in a bead solution and centrifuged to prepare pellets, and then cooled at 4° C. for 10 minutes. Subsequently, the cells were put in a water bath at 37° C., stimulated for 2 minutes and 5 minutes, and after stimulation, cooled on ice for 5 minutes to terminate stimulation. Afterward, the cells were washed with DPBS, and vortexed by adding a lysis buffer, followed by lysis on ice. The resulting cells were then centrifuged, thereby obtaining a cell lysate.

The cell lysate obtained by the above method was subjected to 8% SDS-PAGE using an acryl amide gel, and the protein was transferred to a PVDF membrane (immobilon, #IPVH00010) through semi-dry transfer. Subsequently, after blocking with 5% skim milk, an antigen-antibody reaction was performed. Primary antibodies were treated at 4° C. for 16 hours, and the list of the used primary antibodies is as follows: Phospho-Akt (cell signaling, #CS9271), AKT (cell signaling, #CS9272), Phospho-p44/42 Erkl/2 (cell signaling, #CS9101), and Erk1/2 (cell signaling, #CS4695). Secondary antibodies (mouse anti-rabbit IgG-HRP (Santa Cruz Biotechnology, #SC2357)) were treated at room temperature for 1 hour. Afterward, the protein was detected with ImageQuant LAS 4000 (fujifilm) using a SuperSignal™ WestPico PLUS chemiluminescent substrate (Thermo Scientific, #34580).

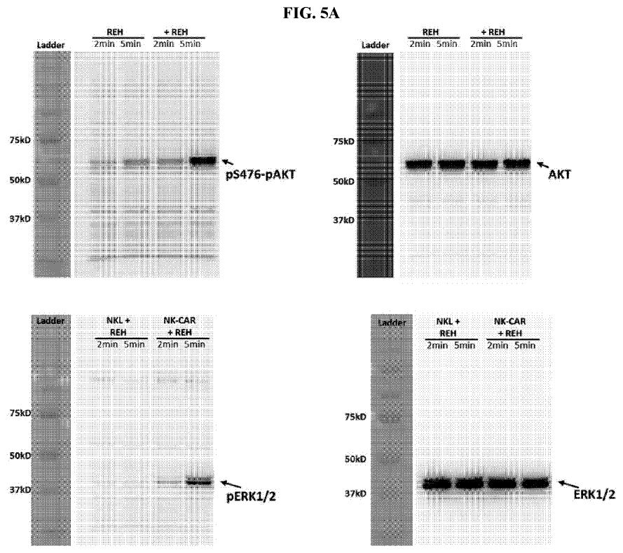

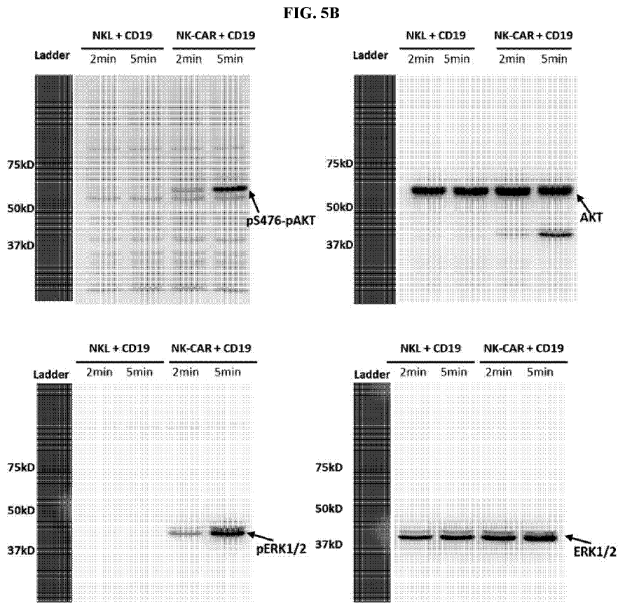

A result of stimulating the NK-CAR (the structure of NK-CAR2 without a CD3ζ cytoplasmic domain)-expressing cell line with CD19-positive REH cells is shown in FIG. 5A. When the same amounts of AKT and ERK were loaded, the level of phosphorylated AKT (pS476-pAKT) to the entire AKT was significantly increased in the NK-CAR-expressing cells stimulated with REH for 5 minutes, and the level of phosphorylated ERK to the total ERK was also significantly increased in the NK-CAR-expressing cells. On the other hand, the control NKL cells, which did not express NK-CAR, had no significant difference in AKT or ERK phosphorylation levels even under stimulation with REH. The result of stimulating the NK-CAR (the structure of NK-CAR2 without a CD3ζ cytoplasmic domain)-expressing cell line with the recombinant human CD19 Fc chimera is shown in FIG. 5B. Like the above result, AKT and ERK signals were not activated by stimulating the NKL cells not expressing NK-CAR with CD19, but when the NK-CAR-expressing NKL cells were stimulated with CD19 for 5 minutes, AKT and ERK phosphorylation was significantly increased.

The above results show that the NK-CAR-expressing NK cells according to the present invention effectively recognize the target antigen CD19, specifically causing AKT and ERK signal activation.

### Example 6. Confirmation of Cytotoxic Degranulation of NK-CAR-Expressing NK Cells

Degranulation is a cell process of releasing an antibacterial cytotoxic material from secretory vesicles called granules. Degranulation is observed in immune cells such as granulocytes (neutrophils, basophils, and eosinophils), mast cells, NK cells, or T cells, and is primarily aimed at attacking invasive microorganisms. Particularly, activated NK cells induce cell death by releasing a material containing perform and granzyme on the surface of a target cell through degranulation. The inner surface of the granules of NK cells was coated with CD107a. After degranulation, CD107a was exposed at the surface of a cytotoxic lymphocyte, so that the outer membrane of lymphocytes was protected from damage caused by perform. The degranulation assay of the NK cells based on the externalization of CD107a allows direct detection of the activation of NK cells responding to an antigen. Accordingly, in this example, the activation of NK cells by NK-CAR was verified by measuring the degree of degranulation of NK-CAR-induced NK cells of the present invention.

Specifically, to evaluate the cytotoxic degranulation of CAR-induced NK92 cells, the CD19-positive cell lines such as REH and Ramos cells, on day 2 of culture were used as target cells, and the NK-CAR-introduced NK92 cells on day 2 of culture, and the 2^{nd} CAR-introduced NK92 cells as a control were used. In addition, pMXs-IRES-GFP-introduced NK92 cells were used as effector cells.

The target cells, the effector cells, and the anti-CD107a/LAMP1-PE (clone H4A3, BD Bioscience, #555801) were suspended in IMDM (+10% FBS, 1% pen/strep) to be 1.0×10⁵ cells/50 μL/sample, 1.0×10⁵ cells/100 μL/sample, and 2 μL/50 μl/sample, respectively, seeded in 96-well plates to have the final volume of 200 μL, and then co-cultured in a CO₂ incubator equilibrated with 5.0% CO₂ gas at 37° C. for 2 hours. Afterward, cell pellets were suspended in DPBS (+1% FBS) by centrifugation, and treated with each of anti-CD3-PerCP (clone SK7, BD Bioscience, #347344), anti-CD56-APC (clone NCAM16.2, BD Bioscience, #341025), and anti-CD107a/LAMP1-PE (clone H4A3, BD Bioscience, #555801) for labeling in a dark room at 4° C. for 35 minutes. Afterward, the cells were washed with DPBS (+1% FBS) twice, and the CD107a expression on the surface of NK92 cells was confirmed through flow cytometry to evaluate cytotoxic degranulation.

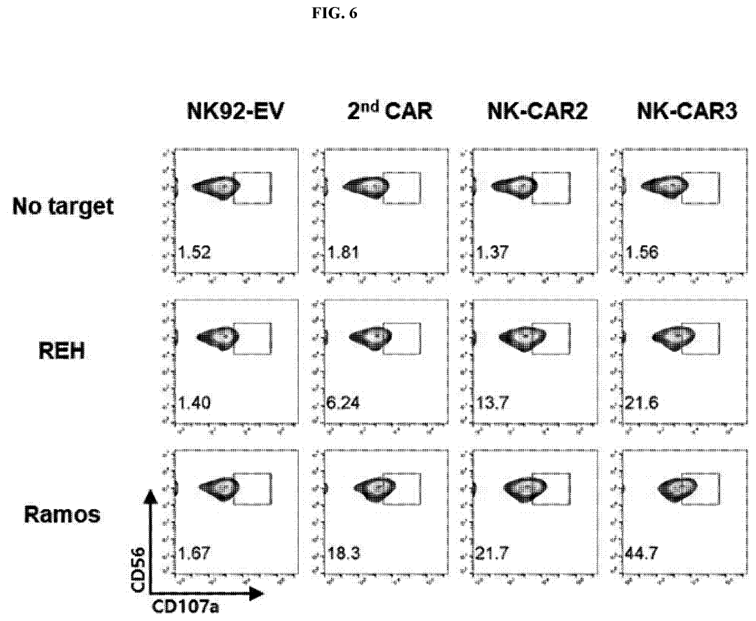

As a result, as shown in FIG. 6, when NK cells were co-cultured with CD19-expressing target cells, it was confirmed that, compared to empty vector (EV)-introduced cells, the proportion of CD107a-positive cells among the CAR-introduced NK92 cells was increased. In addition, as a result of comparing the NK-CAR-introduced NK92 cells and the 2^{nd} CAR-introduced NK92 cells, it was shown that, compared to the 2^{nd} CAR-introduced cells, the degranulation level of the NK-CAR-introduced NK92 cells of the present invention was higher, and particularly, for the NK-CAR3-introduced NK92 cells based on the NK activation receptor combination (NKG2D+2B4), in the REH stimulation group and the Ramos stimulation group, the proportions of CD107a-positive cells were highest at 21.6% and 44.7%, respectively.

The above results show that the NK-CAR of the present invention can more effectively induce the degranulation of NK cells compared to existing CAR, and thus can promote more excellent cytolytic activity.

### Example 7. Confirmation of Degree of Cytokine Production of NK-CAR-Expressing NK Cells

In this example, it was further verified whether the NK-CAR of the present invention activated NK cells and promoted the production of cytokines.

Specifically, to evaluate the cytokine production in NK-CAR-introduced NK92 cells, CD19-positive cell lines, such as REH and Ramos cells, on day 2 of culture were used as target cells, and NK-CAR-introduced NK92 on day 2 of culture and 2^{nd} CAR-introduced NK92 cells as a control were used. In addition, pMXs-IRES-GFP-introduced NK92 cells were used as effector cells.

The target cells and the effector cells were suspended in IMDM (+10% FBS, 1% pen/strep) to be 1.0×10⁵ cells/100 μL/sample and 1.0×10⁵ cells/100 μL/sample, respectively, and seeded in 96-well plates, and then co-cultured in a CO₂ incubator equilibrated with 5.0% CO₂ gas at 37° C. for 1 hour. Afterward, a protein transport inhibitor (GolgiStop; BD Bioscience, #51-2092KZ) containing brefeldin A (GolgiPlug; BD Biosciences, #51-2301KZ) and monensin was added, and further cultured for 5 hours. After a total of 6 hours, cell pellets were suspended in DPBS (+1% FBS) by centrifugation, and labeled with anti-CD3-PerCP (clone SK7, BD Bioscience, #347344), and anti-CD56-APC (clone NCAM16.2, BD Bioscience, #341025) in a dark room at 4° C. for 35 minutes. Afterward, the cells were washed with DPBS (+1% FBS) twice, and fixed/permeabilized with a BD Cytofix/Cytoperm solution (BD Biosciences, #51-2090KZ) in a dark room at 4° C. for 20 minutes. Afterward, the cells were washed twice with 1×BD perm/wash buffer (BD Biosciences, #51-2091KZ), and stained with anti-interferon-γ-PE (clone 25723.11, BD Bioscience, #340452) in a dark room at 4° C. for 15 hours. After staining, the cells were washed with 1×BD perm/wash buffer twice, and the interferon-γ expression of the NK92 cells was confirmed through flow cytometry to evaluate cytokine production.

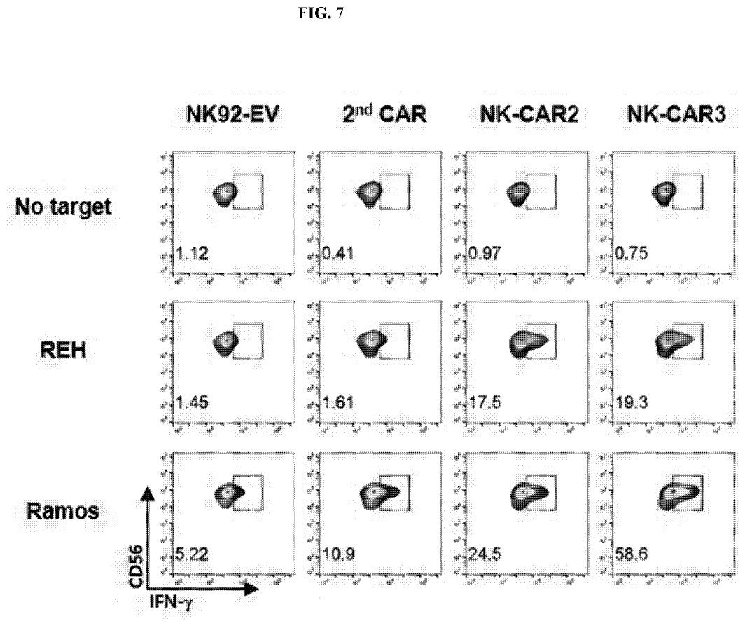

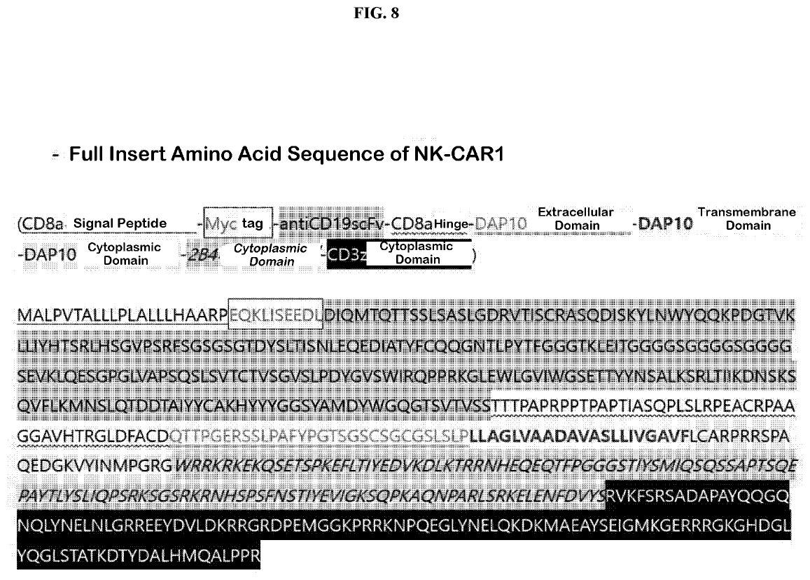

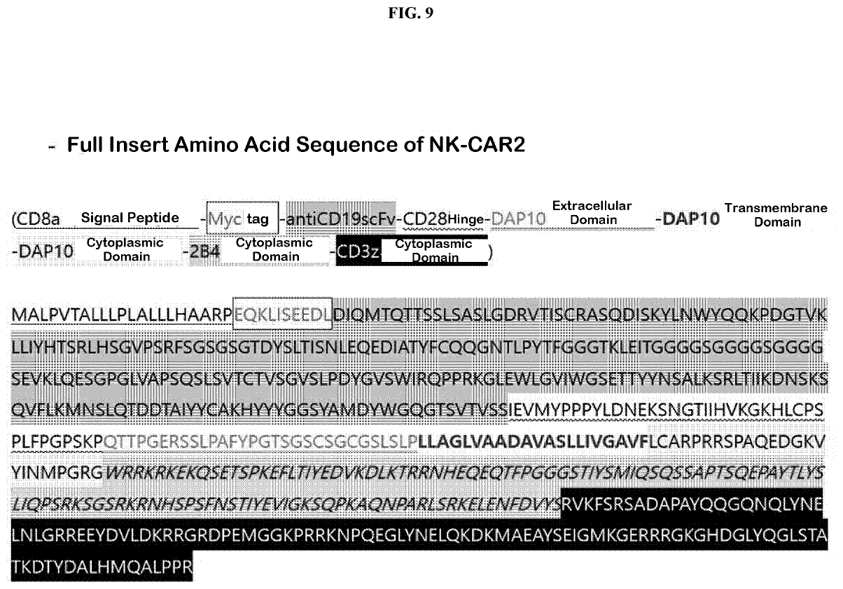

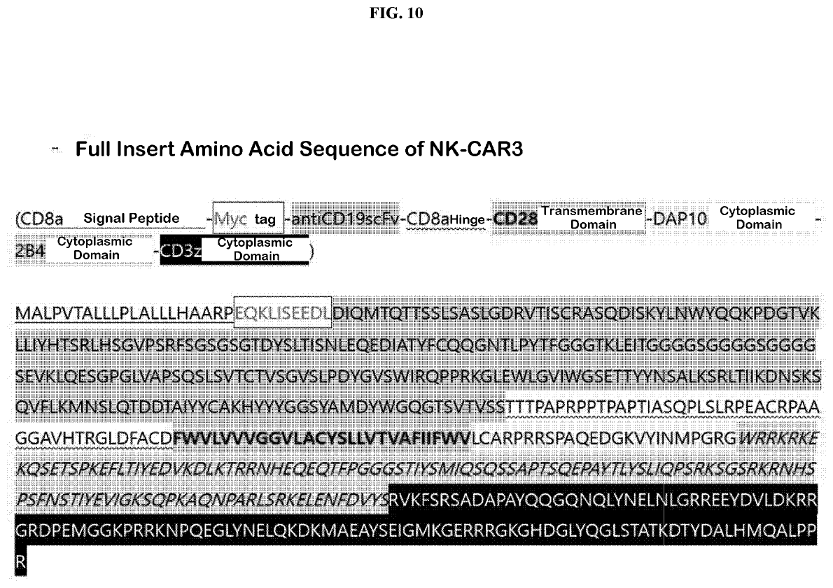

The result is shown in FIG. 7. As a result of confirming the production of interferon-γ in each type of NK92 cells, when co-cultured with the CD19-expressing target cells, compared to the empty vector (EV)-introduced cells, the proportion of the interferon-γ-positive cells among the CAR-introduced NK92 cells was increased. In addition, as a result of comparing the NK-CAR-introduced cells and the 2^{nd} CAR-introduced cells, it was confirmed that, compared to the 2^{nd} CAR-introduced NK92 cells, the proportion of the interferon-γ-positive cells among the NK-CAR-introduced NK92 cells of the present invention was higher, and particularly, for the NK activation receptor combination (NKG2D+2B4)-based NK-CAR3-introduced NK92 cells, the proportions of the interferon-γ-positive cells were the highest of 19.3% and 58.6% in the REH stimulation group and the Ramos stimulation group, respectively.

The results show that the NK-CAR of the present invention can more effectively promote the cytokine production in NK cells than an existing CAR, and thus can induce higher cytolytic activity.

As seen above, the present inventors derived NK-CARs that can exhibit a synergistic NK cell activation effect from the combination of the NK cell-specific activation receptors. The experiments using various NK cells show that the CAR-expressing NK cells effectively recognize target cells, activating AKT and ERK signals, and thus excellent anti-tumor activity was induced. In addition, the CAR activates NK cells to promote the production and secretion of cytotoxic materials such as cytokines, and induces degranulation to enhance cytolytic activity. Particularly, it was confirmed that the NK-CAR of the present invention has higher NK cell activation and cytolytic function induction effects than a T cell-based CAR. Accordingly, the NK-CARs of the present invention, which are novel anticancer immunotherapy means, are expected to reduce the risk of side effects of existing anticancer therapies and exhibit better anticancer effects.

The above description of the present invention is merely provided to exemplify the present invention, and it will be understood by those of ordinary skill in the art to which the present invention belongs that the present invention can be implemented in modified forms without departing from the essential features of the present invention. Therefore, the exemplary embodiments described above should be interpreted as illustrative and not limited in any aspect.

Specific sequence data related to the present invention are shown in Table 1 below.

TABLE 1

| | | SEQ ID |
|---|---|---|
| Classification | Sequence | NO: |
| Anti-CD19 | DIQMTQTTSSLSASLGDRVTISCRASQDISKYLNWYQQKPDGTVKLLIYHTSR | 1 |
| scFv | LHSGVPSRFSGSGSGTDYSLTISNLEQEDIATYFCQQGNTLPYTFGGGTKLEIT | |
| | GGGGSGGGGSGGGGSEVKLQESGPGLVAPSQSLSVTCTVSGVSLPDYGVSWI | |
| | RQPPRKGLEWLGVIWGSETTYYNSALKSRLTIIKDNSKSQVFLKMNSLQTDDT | |
| | AIYYCAKHYYYGGSYAMDYWGQGTSVTVSS | |
| CD8 hinge | TTTPAPRPPTPAPTIASQPLSLRPEACRPAAGGAVHTRGLDFACD | 2 |
| domain | | |
| CD28 hinge | IEVMYPPPYLDNEKSNGTIIHVKGKHLCPSPLFPGPSKP | 3 |
| domain | | |
| CD28 TM | FWVLVVVGGVLACYSLLVTVAFIIFWV | 4 |
| domain | | |
| DAP10 EC | QTTPGERSSLPAFYPGTSGSCSGCGSLSLP | 5 |
| domain | | |
| DAP10 TM | LLAGLVAADAVASLLIVGAVF | 6 |
| domain | | |
| DAP10 CYP | LCARPRRSPAQEDGKVYINMPGRG | 7 |
| domain | | |
| 2B4 CYP | WRRKRKEKQSETSPKEFLTIYEDVKDLKTRRNHEQEQTFPGGGSTIYSMIQSQ | 8 |
| domain | SSAPTSQEPAYTLYSLIQPSRKSGSRKRNHSPSFNSTIYEVIGKSQPKAQNPARL | |
| | SRKELENFDVYS | |
| CD3z CYP | RVKFSRSADAPAYQQGQNQLYNELNLGRREEYDVLDKRRGRDPEMGGKPRR | 9 |
| domain | KNPQEGLYNELQKDKMAEAYSEIGMKGERRRGKGHDGLYQGLSTATKDTY | |
| | DALHMQALPPR | |
| CD8α signal | MALPVTALLLPLALLLHAARP | 10 |
| peptide | | |
| Anti-CD19 | GATATACAGATGACGCAGACAACGTCAAGTCTTTCCGCCAGCTTGGGAGA | 11 |
| scFv | CCGAGTGACTATATCTTGTAGAGCAAGCCAGGATATTTCTAAGTATCTTAA | |
| | CTGGTACCAACAAAAGCCCGATGGAACGGTTAAGCTGCTTATATACCATA | |
| | CCAGTAGACTCCACTCCGGCGTACCATCACGGTTTTCTGGCAGTGGCTCCG | |
| | GGACCGACTATTCTTTGACGATCTCTAATCTCGAACAAGAGGATATTGCA | |
| | ACATACTTTTGTCAGCAAGGCAATACCTTGCCATATACGTTTGGGGGCGG | |
| | GACAAAACTTGAGATAACCGGCGGCGGTGGTTCAGGCGGTGGCGGTTCCG | |
| | GTGGTGGGGGATCAGAGGTTAAGCTTCAGGAATCCGGACCAGGTTTGGTT | |
| | GCCCCCAGCCAATCTCTCAGCGTTACATGCACGGTTTCAGGCGTCAGTCTC | |
| | CCCGATTACGGTGTAAGTTGGATTCGGCAACCTCCGCGAAAGGGTCTGGA | |
| | ATGGCTGGGGGTTATTTGGGGGAGTGAGACAACTTATTACAACTCTGCAC | |
| | TTAAGAGTCGGCTTACCATCATCAAGGATAATTCAAAATCACAAGTATTC | |
| | CTGAAGATGAACTCATTGCAAACAGATGATACAGCTATATACTATTGTGC | |
| | CAAGCATTACTATTATGGTGGTTCTTATGCAATGGATTACTGGGGGCAAG | |
| | GCACGTCAGTGACAGTGAGTTCA | |
| CD8 hinge | ACCACAACCCCAGCACCCCGGCCCCCTACACCTGCACCAACCATCGCCAG | 12 |
| domain_NK- | CCAGCCTCTGTCCCTGAGGCCAGAGGCCTGCCGCCCCGCCGCCGGCGGAG | |
| CAR1 | CAGTGCACACAAGGGGCCTGGACTTCGCCTGTGAT | |
| CD8 hinge | ACCACGACGCCAGCGCCGCGACCACCAACACCGGCGCCCACCATCGCGTC | 13 |
| domain_NK- | GCAGCCCCTGTCCCTGCGCCCAGAGGCGTGCCGGCCAGCGGCGGGGGGCG | |
| CAR3 | CAGTGCACACGAGGGGGCTGGACTTCGCCTGTGAT | |
| CD28 hinge | ATTGAAGTTATGTATCCTCCTCCTTACCTAGACAATGAGAAGAGCAATGG | 14 |
| domain | AACCATTATCCATGTGAAAGGGAAACACCTTTGTCCAAGTCCCCTATTTCC | |
| | CGGACCTTCTAAGCCC | |
| CD28 TM | TTTTGGGTGCTGGTGGTGGTTGGTGGAGTCCTGGCTTGCTATAGCTTGCTA | 15 |
| domain | GTAACAGTGGCCTTTATTATTTTCTGGGTG | |
| DAP10 EC | CAGACAACCCCCGGCGAGCGCAGCTCCCTGCCAGCCTTTTACCCTGGCAC | 16 |
| domain | CTCCGGCTCTTGCAGCGGATGTGGCTCCCTGTCTCTGCCT | |
| DAP10 TM | CTGCTGGCCGGCCTGGTGGCCGCCGACGCAGTGGCCTCTCTGCTGATCGTG | 17 |
| domain | GGAGCCGTGTTC | |
| DAP10 CYP | CTGTGCGCCAGGCCACGGAGATCCCCAGCACAGGAGGATGGCAAGGTGT | 18 |
| domain | ATATCAACATGCCTGGCAGGGGA | |
| 2B4 CYP | TGGCGGAGAAAGCGGAAGGAGAAGCAGAGCGAGACATCCCCTAAGGAGT | 19 |
| domain | TTCTGACCATCTACGAGGACGTGAAGGATCTGAAGACAAGGCGCAATCAC | |
| | GAGCAGGAGCAGACATTCCCCGGCGGAGGCTCCACCATCTATTCTATGAT | |
| | CCAGAGCCAGAGCTCCGCCCCCACATCCCAGGAGCCTGCCTACACCCTGT | |
| | ATTCCCTGATCCAGCCCAGCCGGAAGTCTGGCAGCCGGAAGAGAAACCAC | |
| | TCCCCATCTTTTAATAGCACCATCTACGAAGTGATCGGCAAGTCCCAGCCA | |
| | AAGGCACAGAACCCAGCAAGGCTGTCTCGCAAGGAGCTGGAGAATTTCG | |
| | ACGTGTATAGC | |
| CD3z CYP | AGAGTGAAGTTCAGCAGGAGCGCAGACGCCCCCGCGTACCAGCAGGGCC | 20 |
| domain | AGAACCAGCTCTATAACGAGCTCAATCTAGGACGAAGAGAGGAGTACGA | |
| | TGTTTTGGACAAGAGACGTGGCCGGGACCCTGAGATGGGGGGAAAGCCG | |
| | AGAAGGAAGAACCCTCAGGAAGGCCTGTACAATGAACTGCAGAAAGATA | |
| | AGATGGCGGAGGCCTACAGTGAGATTGGGATGAAAGGCGAGCGCCGGAG | |
| | GGGCAAGGGGCACGATGGCCTTTACCAGGGTCTCAGTACAGCCACCAAGG | |
| | ACACCTACGACGCCCTTCACATGCAGGCCCTGCCCCCTCGC | |
| CD8α signal | ATGGCGCTCCCTGTCACCGCACTGCTTCTTCCGCTGGCACTGCTGCTGCAC | 21 |
| peptide | GCTGCACGGCCT | |
| myc-tag | GAGCAAAAACTTATCTCTGAAGAGGACCTC | 22 |
| NK-CAR1_full | MALPVTALLLPLALLLHAARPEQKLISEEDLDIQMTQTTSSLSASLGDRVTISC | 23 |
| insert | RASQDISKYLNWYQQKPDGTVKLLIYHTSRLHSGVPSRFSGSGSGTDYSLTIS | |
| | NLEQEDIATYFCQQGNTLPYTFGGGTKLEITGGGGSGGGGSGGGGSEVKLQE | |
| | SGPGLVAPSQSLSVTCTVSGVSLPDYGVSWIRQPPRKGLEWLGVIWGSETTYY | |
| | NSALKSRLTIIKDNSKSQVFLKMNSLQTDDTAIYYCAKHYYYGGSYAMDYW | |
| | GQGTSVTVSSTTTPAPRPPTPAPTIASQPLSLRPEACRPAAGGAVHTRGLDFAC | |
| | DQTTPGERSSLPAFYPGTSGSCSGCGSLSLPLLAGLVAADAVASLLIVGAVFLC | |
| | ARPRRSPAQEDGKVYINMPGRGWRRKRKEKQSETSPKEFLTIYEDVKDLKTR | |
| | RNHEQEQTFPGGGSTIYSMIQSQSSAPTSQEPAYTLYSLIQPSRKSGSRKRNHS | |
| | PSFNSTIYEVIGKSQPKAQNPARLSRKELENFDVYSRVKFSRSADAPAYQQGQ | |
| | NQLYNELNLGRREEYDVLDKRRGRDPEMGGKPRRKNPQEGLYNELQKDKM | |
| | AEAYSEIGMKGERRRGKGHDGLYQGLSTATKDTYDALHMQALPPR | |
| NK-CAR2_full | MALPVTALLLPLALLLHAARPEQKLISEEDLDIQMTQTTSSLSASLGDRVTISC | 24 |
| insert | RASQDISKYLNWYQQKPDGTVKLLIYHTSRLHSGVPSRFSGSGSGTDYSLTIS | |
| | NLEQEDIATYFCQQGNTLPYTFGGGTKLEITGGGGSGGGGSGGGGSEVKLQE | |
| | SGPGLVAPSQSLSVTCTVSGVSLPDYGVSWIRQPPRKGLEWLGVIWGSETTYY | |
| | NSALKSRLTIIKDNSKSQVFLKMNSLQTDDTAIYYCAKHYYYGGSYAMDYW | |
| | GQGTSVTVSSIEVMYPPPYLDNEKSNGTIIHVKGKHLCPSPLFPGPSKPQTTPG | |
| | ERSSLPAFYPGTSGSCSGCGSLSLPLLAGLVAADAVASLLIVGAVFLCARPRRS | |
| | PAQEDGKVYINMPGRGWRRKRKEKQSETSPKEFLTIYEDVKDLKTRRNHEQE | |
| | QTFPGGGSTIYSMIQSQSSAPTSQEPAYTLYSLIQPSRKSGSRKRNHSPSFNSTI | |
| | YEVIGKSQPKAQNPARLSRKELENFDVYSRVKFSRSADAPAYQQGQNQLYNE | |
| | LNLGRREEYDVLDKRRGRDPEMGGKPRRKNPQEGLYNELQKDKMAEAYSEI | |
| | GMKGERRRGKGHDGLYQGLSTATKDTYDALHMQALPPR | |
| NK-CAR3_full | MALPVTALLLPLALLLHAARPEQKLISEEDLDIQMTQTTSSLSASLGDRVTISC | 25 |
| insert | RASQDISKYLNWYQQKPDGTVKLLIYHTSRLHSGVPSRFSGSGSGTDYSLTIS | |
| | NLEQEDIATYFCQQGNTLPYTFGGGTKLEITGGGGSGGGGSGGGGSEVKLQE | |
| | SGPGLVAPSQSLSVTCTVSGVSLPDYGVSWIRQPPRKGLEWLGVIWGSETTYY | |
| | NSALKSRLTIIKDNSKSQVFLKMNSLQTDDTAIYYCAKHYYYGGSYAMDYW | |
| | GQGTSVTVSSTTTPAPRPPTPAPTIASQPLSLRPEACRPAAGGAVHTRGLDFAC | |
| | DFWVLVVVGGVLACYSLLVTVAFIIFWVLCARPRRSPAQEDGKVYINMPGRG | |
| | WRRKRKEKQSETSPKEFLTIYEDVKDLKTRRNHEQEQTFPGGGSTIYSMIQSQ | |
| | SSAPTSQEPAYTLYSLIQPSRKSGSRKRNHSPSFNSTIYEVIGKSQPKAQNPARL | |
| | SRKELENFDVYSRVKFSRSADAPAYQQGQNQLYNELNLGRREEYDVLDKRR | |
| | GRDPEMGGKPRRKNPQEGLYNELQKDKMAEAYSEIGMKGERRRGKGHDGL | |
| | YQGLSTATKDTYDALHMQALPPR | |
| Anti-CD19 | GVSLPDYGVS | 26 |
| scFv H | | |
| chain_CDR1 | | |
| Anti-CD19 | VIWGSETTYYNSALKSR | 27 |
| sc Fv H | | |
| chain_CDR2 | | |
| Anti-CD19 | HYYYGGSYAMDY | 28 |
| sc Fv H | | |
| chain_CDR3 | | |
| Anti-CD19 | RASQDISKYLN | 29 |
| scFv L | | |
| chain_CDR1 | | |
| Anti-CD19 | SRLHSGV | 30 |
| sc Fv L | | |
| chain_CDR2 | | |
| Anti-CD19 | QQGNTLPYT | 31 |
| scFv L | | |
| chain_CDR3 | | |

### INDUSTRIAL APPLICABILITY

The present invention relates to an NK cell-specific CAR (NK-CAR), which is completed by confirming that the NK-CAR including a specific combination of NK cell-specific activation receptor-derived domains can effectively activate NK cells and enhance the activity of killing target cells. Specifically, the present inventors prepared three types of NK cell-specific CARs consisting of the combination of specific domains, and confirmed that, in the NK cells expressing the NK-CAR, the production of cytokine and granzyme B responding to target cells or AKT and ERK activation is further enhanced, and degranulation is promoted, resulting in exhibiting better cytolytic activity. That is, since the NK-CAR according to the present invention can enhance cytolytic activity by strongly inducing the activation of NK cells, it can be used in immunotherapy for treating various diseases, including cancer. Particularly, CAR-based anticancer treatment using NK cells cannot cause cytokine release syndrome or neurotoxicity, but can use autologous and allogeneic NK cells, and therefore has an advantage of fewer side effects compared to CAR-T. Therefore, the NK cells that express the NK-CAR according to the present invention are expected to be used as a cellular therapeutic agent with low side effects and a maximized drug treatment effect.

1. A chimeric antigen receptor (CAR), comprising:
(i) an antigen-binding domain;
(ii) a CD8 or CD28 hinge domain;
(iii) a DAP10 cytoplasmic domain;
(iv) a 2B4 cytoplasmic domain; and
(v) a CD3z cytoplasmic domain,
wherein the CAR is expressed in natural killer (NK) cells.

2. The CAR of claim 1, wherein the antigen-binding domain is a tumor antigen-specific antibody or an antigen-binding fragment thereof.

3. The CAR of claim 1, which satisfies one or more of the following characteristics:
(a) the CD8 hinge domain comprises an amino acid sequence of SEQ ID NO: 2;
(b) the CD28 hinge domain comprises an amino acid sequence of SEQ ID NO: 3;
(c) the DAP10 cytoplasmic domain comprises an amino acid sequence of SEQ ID NO: 7;
(d) the 2B4 cytoplasmic domain comprises a amino acid sequence of SEQ ID NO: 8; and
(e) the CD3z cytoplasmic domain comprises a amino acid sequence of SEQ ID NO: 9.

4. The CAR of claim 1, further comprising a CD28 transmembrane domain.

5. The CAR of claim 4, wherein the CD28 transmembrane domain comprises an amino acid sequence of SEQ ID NO: 4.

6. The CAR of claim 1, further comprising one or more selected from the group consisting of a DAP10 extracellular domain and a DAP10 transmembrane domain.

7. The CAR of claim 6, which satisfies one or more of the following characteristics:
(f) the DAP10 extracellular domain comprises an amino acid sequence of SEQ ID NO: 5; and
(g) the DAP10 transmembrane domain comprises a amino acid sequence of SEQ ID NO: 6.

8. The CAR of claim 1, further comprising a signal peptide.

9. The CAR of claim 8, wherein the signal peptide is a CD8 signal peptide.

10. The CAR of claim 2, wherein the tumor antigen is one or more selected from the group consisting of CD19, TAG72, an interleukin 13 receptor alpha-2 subunit (IL13Rα2), CD52, CD33, CD20, TSLPR, CD22, CD30, GD3, CD171, a neural cell adhesion molecule (NCAM), a folate binding protein (FBP), a Lewis-Y antigen (Le(Y)), a prostate stem cell antigen (PSCA), a prostate-specific membrane antigen (PSMA), a carcinoembryonic antigen (CEA), human epidermal growth factor receptor 2 (HER2), mesothelin, hyaluronate receptor variant 6 (CD44v6), B7-H3, Glypican-3, receptor tyrosine kinase-like orphan receptor 1 (ROR1), survivin, folate receptor 1 (FOLR1), Wilm's tumor 1 (WT1), vascular endothelial growth factor 2 (VEGFR2), EGFR, and KRAS.

11. The CAR of claim 2, wherein the antigen-binding fragment is selected from the group consisting of scFv, (scFv)₂, Fab, Fab′, and F(ab′)₂.

12. The CAR of claim 2, wherein the tumor antigen-specific antibody or the antigen-binding fragment thereof comprises a heavy chain variable region and a light chain variable region as follows:
the heavy chain variable region comprises heavy chain CDR1 comprising an amino acid sequence of SEQ ID NO: 26, heavy chain CDR2 comprising an amino acid sequence of SEQ ID NO: 27, and heavy chain CDR3 comprising an amino acid sequence of SEQ ID NO: 28; and
the light chain variable region comprises light chain CDR1 comprising a amino acid sequence of SEQ ID NO: 29, light chain CDR2 comprising an amino acid sequence of SEQ ID NO: 30, and light chain CDR3 comprising an amino acid sequence of SEQ ID NO: 31.

13. A NK cell expressing the CAR of claim 1.

14. The NK cell of claim 13, wherein the NK cell satisfies one or more selected from the group consisting of the following characteristics:
(a) an increased secretion amount of one or more selected from the group consisting of MIP-1α, granzyme B, and INF-γ upon antigen recognition;
(b) an increased activity of one or more selected from the group consisting of an AKT signal and an ERK signal upon antigen recognition; and
(c) an increase in degranulation level upon antigen recognition.

15-17. (canceled)

18. A method for treating cancer, comprising:
administering a therapeutically effective amount of a composition comprising a NK cell that expresses the CAR of claim 1, or a therapeutically effective amount of a cellular therapeutic agent comprising a NK cell that expresses the CAR, to a subject in need thereof.

19. The method of claim 18, wherein the cancer is one or more selected from the group consisting of colorectal cancer, rectal cancer, colon cancer, thyroid cancer, oral cancer, pharynx cancer, larynx cancer, cervical cancer, brain cancer, lung cancer, ovarian cancer, bladder cancer, kidney cancer, liver cancer, pancreatic cancer, prostate cancer, skin cancer, tongue cancer, breast cancer, uterine cancer, stomach cancer, bone cancer, lymphoma, blood cancer, squamous cell carcinoma, adenocarcinoma of the lung, peritoneal cancer, skin melanoma, ocular melanoma, perianal cancer, esophageal cancer, small intestine cancer, endocrine cancer, parathyroid cancer, adrenal cancer, soft tissue sarcoma, urethral cancer, gastrointestinal cancer, glioblastoma, endometrial cancer, salivary gland cancer, vulvar cancer, and head and neck cancer.

20-25. (canceled)

26. The CAR of claim 1, wherein the CAR is prepared by a method comprising:
introducing an expression vector comprising a nucleic acid molecule that encodes the CAR into an isolated cell to form an expression vector-introduced cell; and
culturing the expression vector-introduced cell.

27. The NK cell of claim 13, wherein the NK cell is prepared by a method comprising:
introducing an expression vector comprising a nucleic acid molecule that encodes the CAR into an isolated NK cell.


SEQUENCE LISTING (31 sequences)

SEQ ID NO: 1 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 2 - protein, 45 aa, synthetic construct
TTTPAPRPPTPAPTIASQPLSLRPEACRPAAGGAVHTRGLDFACD

SEQ ID NO: 3 - protein, 39 aa, synthetic construct
IEVMYPPPYLDNEKSNGTIIHVKGKHLCPSPLFPGPSKP

SEQ ID NO: 4 - protein, 27 aa, synthetic construct
FWVLVVVGGVLACYSLLVTVAFIIFWV

SEQ ID NO: 5 - protein, 30 aa, synthetic construct
QTTPGERSSLPAFYPGTSGSCSGCGSLSLP

SEQ ID NO: 6 - protein, 21 aa, synthetic construct
LLAGLVAADAVASLLIVGAVF

SEQ ID NO: 7 - protein, 24 aa, synthetic construct
LCARPRRSPAQEDGKVYINMPGRG

SEQ ID NO: 8 - protein, 120 aa, synthetic construct
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 9 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 21 aa, synthetic construct
MALPVTALLLPLALLLHAARP

SEQ ID NO: 11 - DNA, 726 bp, synthetic construct
  source 1..726: mol_type = other DNA
gatatacagatgacgcagacaacgtcaagtctttccgccagcttgggagaccgagtgactatatcttgtagagcaagccaggatatttctaagtatcttaactggtaccaacaaaagcccgatggaacggttaagctgcttatataccataccagtagactccactccggcgtaccatcacggttttctggcagtggctccgggaccgactattctttgacgatctctaatctcgaacaagaggatattgcaacatacttttgtcagcaaggcaataccttgccatatacgtttgggggcgggacaaaacttgagataaccggcggcggtggttcaggcggtggcggttccggtggtgggggatcagaggttaagcttcaggaatccggaccaggtttggttgcccccagccaatctctcagcgttacatgcacggtttcaggcgtcagtctccccgattacggtgtaagttggattcggcaacctccgcgaaagggtctggaatggctgggggttatttgggggagtgagacaacttattacaactctgcacttaagagtcggcttaccatcatcaaggataattcaaaatcacaagtattcctgaagatgaactcattgcaaacagatgatacagctatatactattgtgccaagcattactattatggtggttcttatgcaatggattactgggggcaaggcacgtcagtgacagtgagttca

SEQ ID NO: 12 - DNA, 135 bp, synthetic construct
  source 1..135: mol_type = other DNA
accacaaccccagcaccccggccccctacacctgcaccaaccatcgccagccagcctctgtccctgaggccagaggcctgccgccccgccgccggcggagcagtgcacacaaggggcctggacttcgcctgtgat

SEQ ID NO: 13 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 14 - DNA, 117 bp, synthetic construct
  source 1..117: mol_type = other DNA
attgaagttatgtatcctcctccttacctagacaatgagaagagcaatggaaccattatccatgtgaaagggaaacacctttgtccaagtcccctatttcccggaccttctaagccc

SEQ ID NO: 15 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 16 - DNA, 90 bp, synthetic construct
  source 1..90: mol_type = other DNA
cagacaacccccggcgagcgcagctccctgccagccttttaccctggcacctccggctcttgcagcggatgtggctccctgtctctgcct

SEQ ID NO: 17 - DNA, 63 bp, synthetic construct
  source 1..63: mol_type = other DNA
ctgctggccggcctggtggccgccgacgcagtggcctctctgctgatcgtgggagccgtgttc

SEQ ID NO: 18 - DNA, 72 bp, synthetic construct
  source 1..72: mol_type = other DNA
ctgtgcgccaggccacggagatccccagcacaggaggatggcaaggtgtatatcaacatgcctggcagggga

SEQ ID NO: 19 - DNA, 360 bp, synthetic construct
  source 1..360: mol_type = other DNA
tggcggagaaagcggaaggagaagcagagcgagacatcccctaaggagtttctgaccatctacgaggacgtgaaggatctgaagacaaggcgcaatcacgagcaggagcagacattccccggcggaggctccaccatctattctatgatccagagccagagctccgcccccacatcccaggagcctgcctacaccctgtattccctgatccagcccagccggaagtctggcagccggaagagaaaccactccccatcttttaatagcaccatctacgaagtgatcggcaagtcccagccaaaggcacagaacccagcaaggctgtctcgcaaggagctggagaatttcgacgtgtatagc

SEQ ID NO: 20 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 21 - DNA, 63 bp, synthetic construct
  source 1..63: mol_type = other DNA
atggcgctccctgtcaccgcactgcttcttccgctggcactgctgctgcacgctgcacggcct

SEQ ID NO: 22 - DNA, 30 bp, synthetic construct
  source 1..30: mol_type = other DNA
gagcaaaaacttatctctgaagaggacctc

SEQ ID NO: 23 - protein, 625 aa, synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDQTTPGERSSLPAFYPGTSGSCSGCGSLSLPLLAGLVAADAVASLLIVGAVFLCARPRRSPAQEDGKVYINMPGRGWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 619 aa, synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPQTTPGERSSLPAFYPGTSGSCSGCGSLSLPLLAGLVAADAVASLLIVGAVFLCARPRRSPAQEDGKVYINMPGRGWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 601 aa, synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDFWVLVVVGGVLACYSLLVTVAFIIFWVLCARPRRSPAQEDGKVYINMPGRGWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 10 aa, synthetic construct
GVSLPDYGVS

SEQ ID NO: 27 - protein, 17 aa, synthetic construct
VIWGSETTYYNSALKSR

SEQ ID NO: 28 - protein, 12 aa, synthetic construct
HYYYGGSYAMDY

SEQ ID NO: 29 - protein, 11 aa, synthetic construct
RASQDISKYLN

SEQ ID NO: 30 - protein, 7 aa, synthetic construct
SRLHSGV

SEQ ID NO: 31 - protein, 9 aa, synthetic construct
QQGNTLPYT